(19) United States
(12) Patent Application Publication
(10) Pub. No.: US 2023/0227424 A1
(43) Pub. Date: Jul. 20, 2023

(54) ULTRAVIOLET ABSORBING AGENT, RESIN COMPOSITION, CURED SUBSTANCE, OPTICAL MEMBER, METHOD OF PRODUCING ULTRAVIOLET ABSORBING AGENT, AND COMPOUND

(71) Applicant: FUJIFILM Corporation, Tokyo (JP)

(72) Inventors: Daisuke SASAKI, Shizuoka (JP); Hidetomo Furuyama, Shizuoka (JP); Yusuke Sakai, Shizuoka (JP); Shinya Hayashi, Shizuoka (JP); Yoshihiro Jimbo, Shizuoka (JP); Atsushi Azuma, Shizuoka (JP)

(73) Assignee: FUJIFILM Corporation, Tokyo (JP)

(21) Appl. No.: 18/177,123

(22) Filed: Mar. 2, 2023

Related U.S. Application Data
(63) Continuation of application No. PCT/JP2021/032668, filed on Sep. 6, 2021.

(30) Foreign Application Priority Data
Sep. 17, 2020 (JP) 2020-156074
Nov. 26, 2020 (JP) 2020-195715

(51) Int. Cl. C07D 339/06; C08K 5/45; G02B 1/04; G02B 5/20

(52) U.S. Cl. CPC C07D 339/06; C08K 5/45; G02B 1/04; G02B 5/208

(57) ABSTRACT

Provided are an ultraviolet absorbing agent including a compound represented by Formula (1), in which the ultraviolet absorbing agent has a maximum absorption wavelength in a wavelength range of 350 to 390 nm in an ethyl acetate solution, and a value obtained by dividing an absorbance at a wavelength of 430 nm by an absorbance at the maximum absorption wavelength is 0.01 or less, a resin composition, a cured substance, and an optical member which include the ultraviolet absorbing agent, a method of producing an ultraviolet absorbing agent, and a compound. In Formula (1), X¹ and X² each independently represent a cyano group or the like, R¹ and R² each independently represent an alkyl group or the like, and R³ and R⁴ each independently represent a hydrogen atom, a halogen atom, an alkyl group, an aryl group, an alkoxy group, or an aryloxy group.

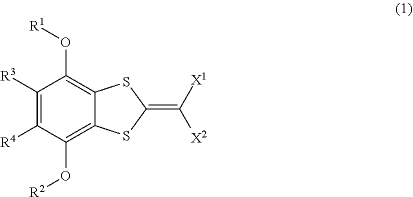

### CROSS-REFERENCE TO RELATED APPLICATIONS

This application is a Continuation of PCT International Application No. PCT/JP2021/032668 filed on Sep. 6, 2021, which claims priority under 35 U.S.C § 119(a) to Japanese Patent Application No. 2020-156074 filed on Sep. 17, 2020, and Japanese Patent Application No. 2020-195715 filed on Nov. 26, 2020. Each of the above application(s) is hereby expressly incorporated by reference, in its entirety, into the present application.

### BACKGROUND OF THE INVENTION

### 1. Field of the Invention

The present invention relates to an ultraviolet absorbing agent. More specifically, the present invention relates to an ultraviolet absorbing agent containing a benzodithiol compound. Further, the present invention relates to a resin composition containing an ultraviolet absorbing agent, a cured substance formed of the resin composition, and an optical member formed of the resin composition. Further, the present invention relates to a method of producing an ultraviolet absorbing agent, and a compound.

### 2. Description of the Related Art

A benzodithiol compound has excellent absorbency of ultraviolet rays and has been used as an ultraviolet absorbing agent or the like. For example, JP1974-011155B (JP-S49-011155B) and JP2009-096971A describe that a specific benzodithiol compound is used as an ultraviolet absorbing agent.

### SUMMARY OF THE INVENTION

Further, the ultraviolet absorbing performance of an ultraviolet absorbing agent may be degraded with time due to irradiation with light. In particular, an ultraviolet absorbing agent having a maximum absorption wavelength on a longer wavelength side in an ultraviolet region has a tendency that the light resistance is poor and the ultraviolet absorption ability thereof is likely to be degraded with time. Therefore, in recent years, there has been a demand for further improvement of light resistance performance of the ultraviolet absorbing agent.

In addition, it is preferable that the fluorescence intensity of the ultraviolet absorbing agent is low.

Therefore, an object of the present invention is to provide an ultraviolet absorbing agent having low fluorescence intensity and excellent light resistance. Further, an object of the present invention is to provide a resin composition, a cured substance, an optical member, a method of producing an ultraviolet absorbing agent, and a compound.

A compound represented by Formula (1) has an excellent absorption ability for light having a wavelength of approximately 350 to 390 nm. However, in a case where the compound is synthesized by a method of the related art, the compound has an absorption at a wavelength of approximately 430 nm and is slightly colored. The absorption has been considered to be caused by the structure of the compound in the related art. As a result of intensive examination on the compound represented by Formula (1), the present inventors have found that in a case where the compound after synthesis is treated by being brought into contact with an adsorbent such as activated carbon or activated alumina, the absorption at a wavelength of approximately 430 nm can be decreased and the coloration can be suppressed. As a result of further examination on the compound represented by Formula (1) in which the absorption at a wavelength of approximately 430 nm has been decreased, the present inventors have found that the light resistance of the compound can be greatly improved and a decrease in ultraviolet absorption ability due to irradiation with light can be greatly suppressed as compared with the compound in a state before the decrease in absorption at a wavelength of approximately 430 nm (hereinafter, also referred to as a crude compound). Further, in a case where the compound represented by Formula (1) in which the absorption at a wavelength of approximately 430 nm has been decreased is irradiated with light having a wavelength of 375 nm and the fluorescence intensity thereof is confirmed, the present inventors have found that the fluorescence intensity of the compound can be greatly reduced as compared with the crude compound. It is usually considered that energy transfer occurs and the fluorescence intensity is decreased in the fluorescence spectrum that can be measured with excitation light having a wavelength of 375 nm in a case where absorption is present on a wavelength side longer than 375 nm. Therefore, it has been usually considered that the fluorescence intensity of the crude compound is lower than that of the compound represented by Formula (1), but the fluorescence intensity of the compound represented by Formula (1) can be greatly reduced by decreasing the absorption at a wavelength of approximately 430 nm. Such an effect is a completely unexpected and surprising effect.

The present invention has been completed based on such examination conducted by the present inventors. The present invention provides the following aspects.

<1> An ultraviolet absorbing agent comprising: a compound represented by Formula (1), in which the ultraviolet absorbing agent has a maximum absorption wavelength in a wavelength range of 350 to 390 nm in an ethyl acetate solution, and a value obtained by dividing an absorbance at a wavelength of 430 nm by an absorbance at the maximum absorption wavelength is 0.01 or less,

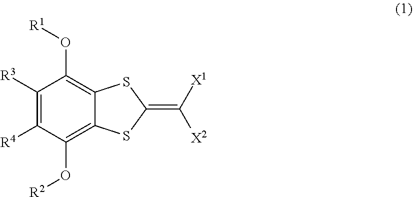

in Formula (1), X¹ and X² each independently represent a hydrogen atom or a substituent, R¹ and R² each independently represent an alkyl group, an acyl group, a carbamoyl group, an aryl group, an alkoxycarbonyl group, or an aryloxycarbonyl group, and R³ and R⁴ each independently represent a hydrogen atom, a halogen atom, an alkyl group, an aryl group, an alkoxy group, or an aryloxy group, where at least one of X¹ or X² represents a substituent having a Hammett's substituent constant σp value of 0.2 or greater.

<2> The ultraviolet absorbing agent according to <1>, in which X¹ and X² in Formula (1) represent a cyano group.

<3> The ultraviolet absorbing agent according to <1> or <2>, in which R¹ and R² in Formula (1) each independently represent a branched alkyl group having 6 or more carbon atoms, and at least one of R³ or R⁴ represents an alkyl group, an alkoxy group, or an aryloxy group.

<4> The ultraviolet absorbing agent according to <1> or <2>, in which R¹ and R² in Formula (1) each independently represent a branched alkyl group having 6 or more carbon atoms, R³ represents an alkyl group, and R⁴ represents a hydrogen atom or an alkyl group.

<5> A resin composition comprising: the ultraviolet absorbing agent according to any one of <1> to <4>; and a resin.

<6> The resin composition according to <5>, in which the resin is at least one selected from a (meth)acrylic resin, a polystyrene resin, a polyester resin, a polyurethane resin, a thiourethane resin, a polyimide resin, an epoxy resin, a polycarbonate resin, or a cellulose acylate resin.

<7> A cured substance which is formed of the resin composition according to <5> or <6>.

<8> An optical member comprising: the ultraviolet absorbing agent according to any one of <1> to <4>.

<9> A method of producing the ultraviolet absorbing agent according to any one of <1> to <4>, the method comprising: reacting a compound represented by Formula (10) with a compound represented by Formula (20) to synthesize a compound represented by Formula (1) and treating the synthesized compound by bringing the compound into contact with an adsorbent,

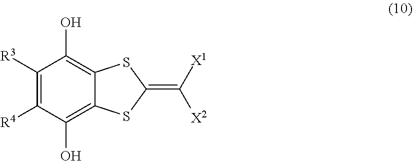

in Formula (10), X¹ and X² each independently represent a hydrogen atom or a substituent, and R³ and R⁴ each independently represent a hydrogen atom, a halogen atom, an alkyl group, an aryl group, an alkoxy group, or an aryloxy group, where at least one of X¹ or X² represents a substituent having a Hammett's substituent constant σp value of 0.2 or greater,

R²¹-E²¹ (20)

in Formula (20), E²¹ represents a group that reacts with a hydroxy group in Formula (10), and R²¹ represents an alkyl group, an acyl group, a carbamoyl group, an aryl group, an alkoxycarbonyl group, or an aryloxycarbonyl group,

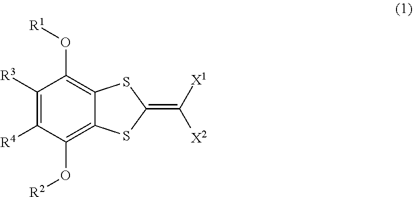

in Formula (1), X¹ and X² each independently represent a hydrogen atom or a substituent, R¹ and R² each independently represent an alkyl group, an acyl group, a carbamoyl group, an aryl group, an alkoxycarbonyl group, or an aryloxycarbonyl group, and R³ and R⁴ each independently represent a hydrogen atom, a halogen atom, an alkyl group, an aryl group, an alkoxy group, or an aryloxy group, where at least one of X¹ or X² represents a substituent having a Hammett's substituent constant σp value of 0.2 or greater.

<10> The method of producing an ultraviolet absorbing agent according to <9>, in which the adsorbent is at least one selected from activated carbon or activated alumina.

<11> A compound which is represented by Formula (1a),

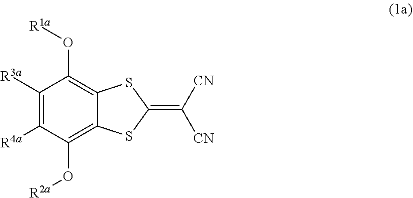

in Formula (1a), R^{1a} and R^{2a} each independently represent a branched alkyl group having 6 or more carbon atoms, R^{3a} represents an alkyl group, and R^{4a} represents a hydrogen atom or an alkyl group.

According to the present invention, it is possible to provide an ultraviolet absorbing agent having low fluorescence intensity and excellent light resistance. Further, according to the present invention, it is possible to provide a resin composition, a cured substance, an optical member, a method of producing a compound, and a compound.

### DESCRIPTION OF THE PREFERRED EMBODIMENTS

Hereinafter, the contents of the present disclosure will be described in detail.

In a case where substitution or unsubstitution is not specified in the notation of a group (atomic group) in the present specification, the group includes both a group which has no substituent and a group which has a substituent. For example, “alkyl group” includes not only an alkyl group having no substituent (unsubstituted alkyl group), but also an alkyl group having a substituent (substituted alkyl group).

In the present specification, a numerical range shown using “to” indicates a range including the numerical values described before and after “to” as the lower limit value and the upper limit value.

In the present specification, the total solid content denotes the total amount of components excluding solvents from all the components of the resin composition.

In the present specification, “(meth)acrylate” denotes both or any one of acrylate and methacrylate, “(meth)acryl” denotes both or any one of acryl and methacryl, “(meth)allyl” denotes both or any one of allyl and methallyl, and “(meth)acryloyl” denotes both or any one of acryloyl and methacryloyl.

In the present specification, the meaning of the term “step” includes not only an independent step but also a step whose intended purpose is achieved even in a case where the step is not clearly distinguished from other steps.

In the present specification, the weight-average molecular weight (Mw) and the number average molecular weight (Mn) are defined as values in terms of polystyrene, measured by gel permeation chromatography (GPC).

<Ultraviolet Absorbing Agent>

An ultraviolet absorbing agent according to the embodiment of the present invention is an ultraviolet absorbing agent containing a compound represented by Formula (1) (hereinafter, also referred to as a compound (1)), and the ultraviolet absorbing agent has a maximum absorption wavelength in a wavelength range of 350 to 390 nm in an ethyl acetate solution, and a value obtained by dividing an absorbance at a wavelength of 430 nm by an absorbance at the maximum absorption wavelength (hereinafter, also referred to as an absorbance ratio 1) is 0.01 or less.

The compound (1) contained in the ultraviolet absorbing agent according to the embodiment of the present invention has an excellent absorption ability for light having a wavelength of approximately 350 to 390 nm. Further, in a case where the ultraviolet absorbing agent according to the embodiment of the present invention contains the compound (1), and the absorbance ratio 1 is 0.01 or less, an ultraviolet absorbing agent with low fluorescence intensity and excellent light resistance can be obtained. The detailed reason why such an effect is obtained is unknown, but it is presumed as follows. After the synthesis of the compound (1), it is presumed that a compound having an absorption at a wavelength of approximately 430 nm is also produced in addition to the compound (1) as impurities. It is presumed that the compound having an absorption at a wavelength of approximately 430 nm is a fluorescent substance having an absorption up to the vicinity of the main absorption of the compound (1). It is presumed that since the ultraviolet absorbing agent according to the embodiment of the present invention contains the compound (1) and the absorbance ratio 1 is 0.01 or less, the content of the fluorescent substance is extremely small. For this reason, it is presumed that the ultraviolet absorbing agent according to the embodiment of the present invention has low fluorescence intensity and excellent light resistance.

The ultraviolet absorbing agent according to the embodiment of the present invention has preferably a maximum absorption wavelength in a wavelength range of 355 to 390 nm and more preferably a maximum absorption wavelength in a wavelength range of 360 to 390 nm in an ethyl acetate solution. In addition, the absorbance ratio 1 is more preferably 0.005 or less.

In the ultraviolet absorbing agent containing the compound (1), the absorbance ratio 1 can be set to 0.01 or less by, for example, a method of treating the compound (1) after synthesis by bringing the compound into contact with an adsorbent. Examples of the adsorbent include a resin-based adsorbent such as an ion exchange resin or a chelate resin, activated carbon, activated alumina, silica gel, zeolite, a hydrotalcite-like compound, and a mixed adsorbent (such as a mixture of zeolite and a ferrocyanide or a mixture of activated carbon and a ferrocyanide), and from the viewpoint of more effectively decreasing the absorbance at a wavelength of approximately 430 nm, activated carbon and activated alumina are preferable, and activated carbon is more preferable.

The content of the compound (1) in the ultraviolet absorbing agent according to the embodiment of the present invention is preferably 95% by mass or greater, more preferably 98% by mass or greater, and still more preferably 99% by mass or greater.

Next, a compound represented by Formula (1) (compound (1)) contained in the ultraviolet absorbing agent will be described.

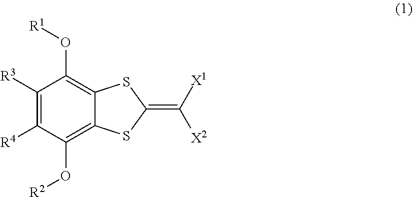

In Formula (1), X¹ and X² each independently represent a hydrogen atom or a substituent, R¹ and R² each independently represent an alkyl group, an acyl group, a carbamoyl group, an aryl group, an alkoxycarbonyl group, or an aryloxycarbonyl group, and R³ and R⁴ each independently represent a hydrogen atom, a halogen atom, an alkyl group, an aryl group, an alkoxy group, or an aryloxy group, where at least one of X¹ or X² represents a substituent having a Hammett's substituent constant σp value of 0.2 or greater.

X¹ and X² each independently represent a hydrogen atom or a substituent. Examples of the substituent include a cyano group, a carbamoyl group, a sulfamoyl group, a nitro group, an acyl group, an alkylsulfonyl group, an arylsulfonyl group, an alkylsulfinyl group, an arylsulfinyl group, an alkoxycarbonyl group, an aryloxycarbonyl group, an alkyl group, an aryl group, and a heterocyclic group. These groups may further have a substituent. Examples of the substituent include groups described in the section of the substituent T below. In a case where a plurality of substituents are provided, the plurality of substituents may be the same as or different from each other. Further, the substituents may be bonded to each other to form a ring.

Examples of the carbamoyl group include a carbamoyl group having 1 to 10 carbon atoms. Among these, a carbamoyl group having 2 to 8 carbon atoms is preferable, and a carbamoyl group having 2 to 5 carbon atoms is more preferable. Specific examples thereof include a methylcarbamoyl group, an ethylcarbamoyl group, and a morpholinocarbonyl group.

Examples of the sulfamoyl group include a sulfamoyl group having 0 to 10 carbon atoms. Among these, a sulfamoyl group having 2 to 8 carbon atoms is preferable, and a sulfamoyl group having 2 to 5 carbon atoms is more preferable. Specific examples thereof include a methylsulfamoyl group, an ethylsulfamoyl group, and a piperidinosulfonyl group.

Examples of the acyl group include an acyl group having 1 to 20 carbon atoms. Among these, an acyl group having 1 to 12 carbon atoms is preferable, and an acyl group having 1 to 8 carbon atoms is more preferable. Specific examples of the acyl group include a formyl group, an acetyl group, a benzoyl group, and a trichloroacetyl group.

Examples of the alkylsulfonyl group include an alkylsulfonyl group having 1 to 20 carbon atoms. Among these, an alkylsulfonyl group having 1 to 10 carbon atoms is preferable, and an alkylsulfonyl group having 1 to 8 carbon atoms is more preferable.

Examples of the arylsulfonyl group include an arylsulfonyl group having 6 to 20 carbon atoms. Among these, an arylsulfonyl group having 6 to 10 carbon atoms is preferable.

Examples of the alkylsulfinyl group include an alkylsulfinyl group having 1 to 20 carbon atoms. Among these, an alkylsulfinyl group having 1 to 10 carbon atoms is preferable, and an alkylsulfinyl group having 1 to 8 carbon atoms is more preferable.

Examples of the arylsulfinyl group include an arylsulfinyl group having 6 to 20 carbon atoms. Among these, an arylsulfinyl group having 6 to 10 carbon atoms is preferable.

Examples of the alkoxycarbonyl group include an alkoxycarbonyl group having 2 to 20 carbon atoms. Among these, an alkoxycarbonyl group having 2 to 12 carbon atoms is preferable, and an alkoxycarbonyl group having 2 to 8 carbon atoms is more preferable. Specific examples thereof include a methoxycarbonyl group, an ethoxycarbonyl group, and a benzyloxycarbonyl group.

Examples of the aryloxycarbonyl group include an aryloxycarbonyl group having 6 to 20 carbon atoms. Among these, an aryloxycarbonyl group having 6 to 12 carbon atoms is preferable, and an aryloxycarbonyl group having 6 to 8 carbon atoms is more preferable. Specific examples thereof include a phenoxycarbonyl group.

Examples of the alkyl group include an alkyl group having 1 to 18 carbon atoms. Among these, an alkyl group having 1 to 10 carbon atoms is preferable, and an alkyl group having 1 to 5 carbon atoms is more preferable. Specific examples thereof include a methyl group, an ethyl group, a propyl group, a butyl group, a hydroxymethyl group, a trifluoromethyl group, a benzyl group, a carboxyethyl group, an ethoxycarbonylmethyl group, an acetylaminomethyl group, an ethoxycarbonylpropyl group, an ethoxycarbonylpentyl group, a butoxycarbonylpropyl group, and a 2-ethylhexyloxycarbonylpropyl group.

Examples of the aryl group include an aryl group having 6 to 20 carbon atoms. Among these, an aryl group having 6 to 15 carbon atoms is preferable, and an aryl group having 6 to 10 carbon atoms is more preferable. Specific examples thereof include a phenyl group, a naphthyl group, a p-carboxyphenyl group, a p-nitrophenyl group, a 3,5-dichlorophenyl group, a p-cyanophenyl group, an m-fluorophenyl group, a p-tolyl group, and a p-bromophenyl group.

It is preferable that the heterocyclic ring in the heterocyclic group includes a 5- or 6-membered saturated or unsaturated heterocyclic ring. The heterocyclic ring may be fused with an aliphatic ring, an aromatic ring, or another heterocyclic ring. Examples of the heteroatom constituting the ring of the heterocyclic ring include B, N, O, S, Se, and Te. Among these, N, O and S are preferable. It is preferable that the carbon atom of the heterocyclic ring has a free valence (monovalent) (the heterocyclic group is bonded at the carbon atom). The number of carbon atoms of the heterocyclic group is preferably in a range of 1 to 40, more preferably in a range of 1 to 30, and still more preferably in a range of 1 to 20. Examples of the saturated heterocyclic ring in the heterocyclic group include a pyrrolidine ring, a morpholine ring, a 2-bora-1,3-dioxolane ring, and a 1,3-thiazolidine ring. Examples of the unsaturated heterocyclic ring in the heterocyclic group include an imidazole ring, a thiazole ring, a benzothiazole ring, a benzoxazole ring, a benzotriazole ring, a benzoselenazole ring, a pyridine ring, a pyrimidine ring, and a quinoline ring.

In Formula (1), at least one of X¹ or X² represents a substituent having a Hammet's substituent constant σp value of 0.2 or greater, and it is preferable that both X¹ and X² represent a substituent having a Hammet's substituent constant σp value of 0.2 or greater.

The Hammet's substituent constant σp value will be described. Hammett's rule is an experimental rule proposed by L. P. Hammett in 1935 to quantitatively discuss the effects of substituents on the reaction or equilibrium of a benzene derivative, and the validity thereof is widely accepted today. The substituent constants acquired by Hammett's rule include a σp value and a am value, and these values can be found in many general books. For example, these values are specifically described in “Lange's Handbook of Chemistry”, edited by J. A. Dean, 12th edition, 1979 (McGraw-Hill), “Chemistry Region”, extra edition, No. 122, pp. 96 to 103, 1979 (Nankodo, Co., Ltd.), and Chem. Rev., 1991, Vol. 91, pp. 165 to 195. In the present specification, a substituent having a Hammett's substituent constant σp value of 0.2 or greater denotes an electron-withdrawing group.

The Hammett's substituent constant σp value of the substituent represented by X¹ and X² is preferably 0.25 or greater, more preferably 0.3 or greater, and still more preferably 0.35 or greater.

Specific examples of the substituent having a Hammet's substituent constant σp value of 0.2 or greater include a cyano group (σp value=0.66), a carboxyl group (—COOH: σp value=0.45), an alkoxycarbonyl group (—COOMe: σp value=0.45), an aryloxycarbonyl group (—COOPh: σp value=0.44), a carbamoyl group (—CONH₂: σp value=0.36), an alkylcarbonyl group (—COMe: σp value=0.50), an arylcarbonyl group (—COPh: σp value=0.43), an alkylsulfonyl group (—SO₂Me: σp value=0.72), and an arylsulfonyl group (—SO₂Ph: σp value=0.68). Me represents a methyl group, and Ph represents a phenyl group. In addition, the values in parentheses are σp values of representative substituents, which are extracted from Chem. Rev., 1991, Vol. 91, pp. 165 to 195.

It is preferable that X¹ and X² in Formula (1) each independently represent a cyano group, a carbamoyl group, a sulfamoyl group, an acyl group, an alkylsulfonyl group, an arylsulfonyl group, an alkylsulfinyl group, an arylsulfinyl group, an alkoxycarbonyl group, or an aryloxycarbonyl group. Here, it is preferable that at least one of X¹ or X² represents a cyano group and more preferable that X¹ and X² represent a cyano group.

R¹ and R² each independently represent an alkyl group, an acyl group, a carbamoyl group, an aryl group, an alkoxycarbonyl group, or an aryloxycarbonyl group and preferably an alkyl group.

Examples of the alkyl group represented by R¹ and R² include an alkyl group having 1 to 30 carbon atoms. The alkyl group may be linear, branched, or cyclic, but is preferably a linear or branched alkyl group and more preferably a branched alkyl group from the viewpoint of improving the solubility and the compatibility with a resin. Among these, from the viewpoint of improving the solubility and the compatibility with a resin, a branched alkyl group having 6 or more carbon atoms is preferable, and a branched alkyl group having 7 or more carbon atoms is more preferable as the alkyl group. The upper limit of the number of carbon atoms in the branched alkyl group is preferably 30 or less, more preferably 20 or less, and still more preferably 15 or less.

The alkyl group represented by R¹ and R² may have a substituent. Examples of the substituent include the groups described in the section of the substituent T below. Among these, an alkoxy group, an aryloxy group, an acyl group, an acyloxy group, an alkoxycarbonyl group, and an aryloxycarbonyl group are preferable, and an alkoxycarbonyl group is more preferable.

Specific examples of the alkyl group represented by R¹ and R² include a methyl group, an ethyl group, an n-propyl group, an isopropyl group, a sec-butyl group, a tert-butyl group, an n-hexyl group, an n-octyl group, an n-decyl group, an eicosyl group, a 2-chloroethyl group, a 2-cyanoethyl group, a benzyl group, a 2-ethylbutyl group, a 2-ethylhexyl group, a 3,5,5-trimethylhexyl group, a 2-hexyldecyl group, a 2-octyldecyl group, a 2-(4,4-dimethylpentane-2-yl)-5,7,7-trimethyloctyl group, an isostearyl group, an isopalmityl group, a vinyl group, an allyl group, a prenyl group, a geranyl group, an oleyl group, a propargyl group, a cyclohexyl group, a cyclopentyl group, an ethoxycarbonylpropyl group, an ethoxycarbonylpentyl group, a butoxycarbonylpropyl group, and a 2-ethylhexyloxycarbonylpropyl group.

An acyl group having 2 to 30 carbon atoms is preferable as the acyl group represented by R¹ and R². The acyl group represented by R¹ and R² may have a substituent. Examples of the substituent include the groups described in the section of the substituent T below. Specific examples of the acyl group include an acetyl group, a pivaloyl group, a 2-ethylhexanoyl group, a stearoyl group, a benzoyl group, and a paramethoxyphenylcarbonyl group.

A carbamoyl group having 1 to 30 carbon atoms is preferable as the carbamoyl group represented by R¹ and R². The carbamoyl group represented by R¹ and R² may have a substituent. Examples of the substituent include the groups described in the section of the substituent T below. Specific examples of the carbamoyl group include a N,N-dimethylcarbamoyl group, a N,N-diethylcarbamoyl group, a morpholinocarbonyl group, a N,N-di-n-octylaminocarbonyl group, and a N-n-octylcarbamoyl group.

As the aryl group represented by R¹ and R², an aryl group having 6 to 30 carbon atoms is preferable, and an aryl group having 6 to 10 carbon atoms is more preferable. The aryl group represented by R¹ and R² may have a substituent. Examples of the substituent include the groups described in the section of the substituent T below. Specific examples of the aryl group include a phenyl group, a p-tolyl group, a naphthyl group, a meta-chlorophenyl group, and an orthohexadecanoylaminophenyl group. A phenyl group is preferable as the aryl group.

Examples of the alkoxycarbonyl group represented by R¹ and R² include an alkyl group having 2 to 30 carbon atoms. The alkoxycarbonyl group represented by R¹ and R² may have a substituent. Examples of the substituent include the groups described in the section of the substituent T below.

Examples of the aryloxycarbonyl group represented by R¹ and R² include an alkyl group having 7 to 30 carbon atoms. The aryloxycarbonyl group represented by R¹ and R² may have a substituent. Examples of the substituent include the groups described in the section of the substituent T below.

R³ and R⁴ in Formula (1) each independently represent a hydrogen atom, a halogen atom, an alkyl group, an aryl group, an alkoxy group, or an aryloxy group.

Examples of the halogen atom include a fluorine atom, a chlorine atom, and a bromine atom.

The alkyl group is preferably an alkyl group having 1 to 30 carbon atoms, more preferably an alkyl group having 1 to 10 carbon atoms, still more preferably an alkyl group having 1 to 5 carbon atoms, and particularly preferably an alkyl group having 1 or 2 carbon atoms. The alkyl group is preferably a linear or branched alkyl group and more preferably a linear alkyl group. The alkyl group may have a substituent. Examples of the substituent include the groups described in the section of the substituent T below. Specific examples of the alkyl group include a methyl group, an ethyl group, an n-propyl group, an isopropyl group, a tert-butyl group, an n-octyl group, a 2-cyanoethyl group, a benzyl group, a 2-ethylhexyl group, a vinyl group, an allyl group, a prenyl group, a geranyl group, an oleyl group, a propargyl group, a cyclohexyl group, a cyclopentyl group, a 2-hydroxyethyl group, and a 2-hydroxypropyl group. Among these, a methyl group and a tert-butyl group are preferable, and a methyl group is more preferable from the viewpoint of ease of synthesis.

The aryl group is preferably an aryl group having 6 to 30 carbon atoms and more preferably an aryl group having 6 to 10 carbon atoms. The aryl group may have a substituent. Examples of the substituent include the groups described in the section of the substituent T below. Specific examples of the aryl group include a phenyl group, a p-tolyl group, and a naphthyl group.

The alkoxy group is preferably an alkoxy group having 1 to 30 carbon atoms. The alkoxy group may have a substituent. Examples of the substituent include the groups described in the section of the substituent T below. Specific examples of the alkoxy group include a methoxy group and an ethoxy group.

An aryloxy group is preferably an aryloxy group having 6 to 30 carbon atoms. The aryloxy group may have a substituent. Examples of the substituent include the groups described in the section of the substituent T below. Specific examples of the aryloxy group include a phenoxy group, a 2-methylphenoxy group, a 4-tert-butylphenoxy group, a 3-nitrophenoxy group, and a 2-tetradecanoylaminophenoxy group.

It is preferable that R³ and R⁴ in Formula (1) each independently represent a hydrogen atom, an alkyl group, an alkoxy group, or an aryloxy group. Further, at least one of R³ or R⁴ represents preferably an alkyl group, an alkoxy group, or an aryloxy group and more preferably an alkyl group. Among these, it is more preferable that R³ represents an alkyl group and R⁴ represents a hydrogen atom or an alkyl group and particularly preferable that R³ represents an alkyl group and R⁴ represents a hydrogen atom.

It is preferable that the compound (1) is a compound represented by Formula (1a) (hereinafter, also referred to as a compound (1a)). The compound (1a) is a compound according to the embodiment of the present invention. Further, the compound (1a) has satisfactory compatibility with a resin or the like and can suppress surface unevenness or the like of a surface of a cured substance. The detailed reason why such an effect is obtained is unknown, but it is presumed that the compound (1a) is likely to be twisted between R^{1a} and R^{3a} due to the influence of steric repulsion and the like. It is presumed that occurrence of such twisting leads to a decrease in the crystallinity of the compound and improvement of compatibility with a resin or the like.

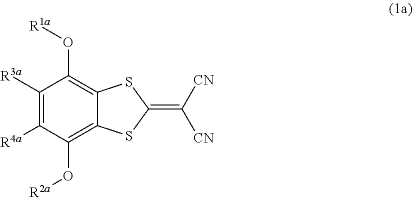

In Formula (1a), R^{1a} and R^{2a} each independently represent a branched alkyl group having 6 or more carbon atoms, R^{3a} represents an alkyl group, and R^{4a} represents a hydrogen atom or an alkyl group.

R^{1a} and R^{2a} in Formula (1a) each independently represent a branched alkyl group having 6 or more carbon atoms and preferably a branched alkyl group having 7 or more carbon atoms. The upper limit of the number of carbon atoms in the branched alkyl group is preferably 30 or less, more preferably 20 or less, and still more preferably 15 or less.

As the alkyl group represented by R^{3a} and R^{4a} in Formula (1a), an alkyl group having 1 to 30 carbon atoms is preferable, an alkyl group having 1 to 10 carbon atoms is more preferable, an alkyl group having 1 to 5 carbon atoms is still more preferable, and an alkyl group having 1 or 2 carbon atoms is particularly preferable. The alkyl group is preferably a linear or branched alkyl group and more preferably a linear alkyl group. Specific examples of the alkyl group include a methyl group, an ethyl group, an n-propyl group, an isopropyl group, a tert-butyl group, an n-octyl group, a 2-cyanoethyl group, a benzyl group, a 2-ethylhexyl group, a vinyl group, an allyl group, a prenyl group, a geranyl group, an oleyl group, a propargyl group, a cyclohexyl group, a cyclopentyl group, a 2-hydroxyethyl group, and a 2-hydroxypropyl group. Among these, a methyl group and a tert-butyl group are preferable, and a methyl group is more preferable.

From the viewpoint of ease of synthesis, it is preferable that R^{4a} in Formula (1a) represents a hydrogen atom.

(Substituent T)

Examples of the substituent T include the following groups.

Examples thereof include a halogen atom (such as a chlorine atom, a bromine atom, or an iodine atom), an alkyl group [a linear, branched, or cyclic alkyl group, specific examples thereof include a linear or branched alkyl group (preferably a linear or branched alkyl group having 1 to 30 carbon atoms, and examples thereof include a methyl group, an ethyl group, an n-propyl group, an isopropyl group, a t-butyl group, an n-octyl group, an eicosyl group, a 2-chloroethyl group, a 2-cyanoethyl group, and a 2-ethylhexyl group), a cycloalkyl group (preferably a cycloalkyl group having 3 to 30 carbon atoms, and examples thereof include a cyclohexyl group, a cyclopentyl group, and a 4-n-dodecylcyclohexyl group), a bicycloalkyl group (preferably a bicycloalkyl group having 5 to 30 carbon atoms, that is, a monovalent group obtained by removing one hydrogen atom from a bicycloalkane having 5 to 30 carbon atoms, and examples thereof include a bicyclo[1,2,2]heptane-2-yl group and a bicyclo[2,2,2]octane-3-yl group), and those having a tricyclo structure with a plurality of ring structures, and alkyl groups in the substituents described below (for example, an alkyl group in an alkylthio group) are alkyl groups of such a concept], an alkenyl group [linear, branched, or cyclic alkenyl group, specific examples thereof include a linear or branched alkenyl group (preferably a linear or branched alkenyl group having 2 to 30 carbon atoms, and examples thereof include a vinyl group, an allyl group, a prenyl group, a geranyl group, and an oleyl group), a cycloalkenyl group (preferably a cycloalkenyl group having 3 to 30 carbon atoms, that is, a monovalent group obtained by removing one hydrogen atom from a cycloalkene having 3 to 30 carbon atoms, and examples thereof include a 2-cyclopentene-1-yl group and a 2-cyclohexene-1-yl group), and a bicycloalkenyl group (preferably a bicycloalkenyl group having 5 to 30 carbon atoms, that is, a monovalent group obtained by removing one hydrogen atom from a bicycloalkene having one double bond, and examples thereof include a bicyclo[2,2,1]hepto-2-en-1-yl group and a bicyclo[2,2,2]octo-2-en-4-yl group)], an alkynyl group (preferably a linear or branched alkynyl group having 2 to 30 carbon atoms, examples thereof include an ethynyl group and a propargyl group),

an aryl group (preferably an aryl group having 6 to 30 carbon atoms, examples thereof include a phenyl group, a p-tolyl group, a naphthyl group, an m-chlorophenyl group, an o-hexadecanoylaminophenyl group), a heterocyclic group (preferably a monovalent group obtained by removing one hydrogen atom from a 5- or 6-membered aromatic or non-aromatic heterocyclic compound and more preferably a 5- or 6-membered aromatic heterocyclic group having 1 to 20 carbon atoms, and examples thereof include a 2-furyl group, a 2-thienyl group, a 2-pyrimidinyl group, and a 2-benzothiazolyl group), a cyano group, a hydroxy group, a nitro group, a carboxyl group, an alkoxy group (preferably a linear or branched alkoxy group having 1 to 30 carbon atoms, and examples thereof include a methoxy group, an ethoxy group, an isopropoxy group, a t-butoxy group, an n-octyloxy group, and a 2-methoxyethoxy group), an aryloxy group (preferably an aryloxy group having 6 to 30 carbon atoms, and examples thereof include a phenoxy group, a 2-methylphenoxy group, a 4-t-butylphenoxy group, a 3-nitrophenoxy group, and a 2-tetradecanoylaminophenoxy group), a heterocyclic oxy group (preferably a heterocyclic oxy group having 2 to 30 carbon atoms, and examples thereof include a 1-phenyltetrazole-5-oxy group and a 2-tetrahydropyranyloxy group), an acyloxy group (preferably a formyloxy group, an alkylcarbonyloxy group having 2 to 30 carbon atoms, or an arylcarbonyloxy group having 6 to 30 carbon atoms, and examples thereof include a formyloxy group, an acetyloxy group, a pivaloyloxy group, a stearoyloxy group, a benzoyloxy group, and a p-methoxyphenylcarbonyloxy group),

a carbamoyloxy group (preferably a carbamoyloxy group having 1 to 30 carbon atoms, and examples thereof include a N,N-dimethylcarbamoyloxy group, a N,N-diethylcarbamoyloxy group, a morpholinocarbonyloxy group, a N,N-di-n-octylaminocarbonyloxy group, and a N-n-octylcarbamoyloxy group), an alkoxycarbonyloxy group (preferably an alkoxycarbonyloxy group having 2 to 30 carbon atoms, and examples thereof include a methoxycarbonyloxy group, an ethoxycarbonyloxy group, a t-butoxycarbonyloxy group, and an n-octylcarbonyloxy group), an aryloxycarbonyloxy group (preferably an aryloxycarbonyloxy group having 7 to 30 carbon atoms, and examples thereof include a phenoxycarbonyloxy group, a p-methoxyphenoxycarbonyloxy group, and a p-n-hexadecyloxyphenoxycarbonyloxy group), an amino group (preferably an amino group, an alkylamino group having 1 to 30 carbon atoms, or an anilino group having 6 to 30 carbon atoms, and examples thereof include an amino group, a methylamino group, a dimethylamino group, an anilino group, a N-methyl-anilino group, and a diphenylamino group), an acylamino group (preferably a formylamino group, an alkylcarbonylamino group having 2 to 30 carbon atoms, or an arylcarbonylamino group having 6 to 30 carbon atoms, and examples thereof include a formylamino group, an acetylamino group, a pivaloylamino group, a lauroylamino group, a benzoylamino group, and a 3,4,5-tri-n-octyloxyphenylcarbonylamino group),

an aminocarbonylamino group (preferably an aminocarbonylamino group having 1 to 30 carbon atoms, and examples thereof include a carbamoylamino group, a N,N-dimethylaminocarbonylamino group, a N,N-diethylaminocarbonylamino group, and a morpholinocarbonylamino group), an alkoxycarbonylamino group (preferably an alkoxycarbonylamino group having 2 to 30 carbon atoms, and examples thereof include a methoxycarbonylamino group, an ethoxycarbonylamino group, a t-butoxycarbonylamino group, an n-octadecyloxycarbonylamino group, and a N-methyl-methoxycarbonylamino group), an aryloxycarbonylamino group (preferably an aryloxycarbonylamino group having 7 to 30 carbon atoms, and examples thereof include a phenoxycarbonylamino group, a p-chlorophenoxycarbonylamino group, and an m-n-octyloxyphenoxycarbonylamino group), a sulfamoylamino group (preferably a sulfamoylamino group having 0 to 30 carbon atoms, and examples thereof include a sulfamoylamino group, a N,N-dimethylaminosulfonylamino group, and a N-n-octylaminosulfonylamino group), an alkyl or arylsulfonylamino group (preferably an alkyl sulfonylamino group having 1 to 30 carbon atoms or an arylsulfonylamino group having 6 to 30 carbon atoms, and examples thereof include a methylsulfonylamino group, a butylsulfonylamino group, a phenylsulfonylamino group, a 2,3,5-trichlorophenylsulfonylamino group, and a p-methylphenylsulfonylamino group), a mercapto group, an alkylthio group (preferably an alkylthio group having 1 to 30 carbon atoms, and examples thereof include a methylthio group, an ethylthio group, and an n-hexadecylthio group), an arylthio group (preferably an arylthio group having 6 to 30 carbon atoms, and examples thereof include a phenylthio group, a p-chlorophenylthio group, and an m-methoxyphenylthio group), a heterocyclic thio group (preferably a heterocyclic thio group having 2 to 30 carbon atoms, and examples thereof include a 2-benzothiazolylthio group and a 1-phenyltetrazole-5-ylthio group),

a sulfamoyl group (preferably a sulfamoyl group having 0 to 30 carbon atoms, and examples thereof include a N-ethylsulfamoyl group, a N-(3-dodecyloxypropyl)sulfamoyl group, a N,N-dimethylsulfamoyl group, a N-acetylsulfamoyl group, a N-benzoylsulfamoyl group, a N—(N′-phenylcarbamoyl)sulfamoyl group), a sulfo group, an alkyl or arylsulfinyl group (preferably an alkyl sulfinyl group having 1 to 30 carbon atoms or an arylsulfinyl group having 6 to 30 carbon atoms, and examples thereof include a methylsulfinyl group, an ethylsulfinyl group, a phenylsulfinyl group, and a p-methylphenylsulfinyl group), an alkyl or arylsulfonyl group (preferably an alkylsulfonyl group having 1 to 30 carbon atoms or an arylsulfonyl group having 6 to 30 carbon atoms, and examples thereof include a methylsulfonyl group, an ethylsulfonyl group, a phenylsulfonyl group, and a p-methylphenylsulfonyl group),

an acyl group (preferably a formyl group, an alkylcarbonyl group having 2 to 30 carbon atoms, an arylcarbonyl group having 7 to 30 carbon atoms, or a heterocyclic carbonyl group having 4 to 30 carbon atoms and bonded to a carbonyl group, and examples include an acetyl group, a pivaloyl group, a 2-chloroacetyl group, a stearoyl group, a benzoyl group, a p-n-octyloxyphenylcarbonyl group, a 2-pyridylcarbonyl group, and a 2-furylcarbonyl group), an aryloxycarbonyl group (preferably an aryloxycarbonyl group having 7 to 30 carbon atoms, and examples thereof include a phenoxycarbonyl group, an o-chlorophenoxycarbonyl group, an m-nitrophenoxycarbonyl group, and a p-t-butylphenoxycarbonyl group), an alkoxycarbonyl group (preferably an alkoxycarbonyl group having 2 to 30 carbon atoms, and examples thereof include a methoxycarbonyl group, an ethoxycarbonyl group, a t-butoxycarbonyl group, an n-octadecyloxycarbonyl group, an n-butoxycarbonyl group, and a 2-ethylhexyloxycarbonyl group), a carbamoyl group (preferably a carbamoyl group having 1 to 30 carbon atoms, and examples thereof include a carbamoyl group, a N-methylcarbamoyl group, a N,N-dimethylcarbamoyl group, a N,N-di-n-octylcarbamoyl group, and a N-(methylsulfonyl)carbamoyl group), an aryl or heterocyclic azo group (preferably an arylazo group having 6 to 30 carbon atoms or a heterocyclic azo group having 3 to 30 carbon atoms, and examples thereof include a phenylazo group, a p-chlorophenylazo group, and a 5-ethylthio-1,3,4-thiadiazole-2-ylazo group), an imide group (preferably a N-succinimide group or a N-phthalimide group), a phosphino group (preferably a phosphino group having 2 to 30 carbon atoms, and examples thereof include a dimethylphosphino group, a diphenylphosphino group, and a methylphenoxyphosphino group), a phosphinyl group (preferably a phosphinyl group having 2 to 30 carbon atoms, and examples thereof include a phosphinyl group, a dioctyloxyphosphinyl group, and a diethoxyphosphinyl group), a phosphinyloxy group (preferably a phosphinyloxy group having 2 to 30 carbon atoms, and examples thereof include a diphenoxyphosphinyloxy group and a dioctyloxyphosphinyloxy group), and a phosphinylamino group (preferably a phosphinylamino group having 2 to 30 carbon atoms, and examples thereof include a dimethoxyphosphinylamino group and a dimethylaminophosphinylamino group).

Among the groups described above, one or more hydrogen atoms of groups having hydrogen atoms may be substituted with the above-described substituents T. Examples of such substituents include an alkylcarbonylaminosulfonyl group, an arylcarbonylaminosulfonyl group, an alkylsulfonylaminocarbonyl group, and an arylsulfonylaminocarbonyl group. Specific examples include a methylsulfonylaminocarbonyl group, a p-methylphenylsulfonylaminocarbonyl group, an acetylaminosulfonyl group, and a benzoylaminosulfonyl group.

Specific examples of the compound (1) include compounds having the following structures. In the structural formulae shown below, Me represents a methyl group, and tBu represents a tert-butyl group.

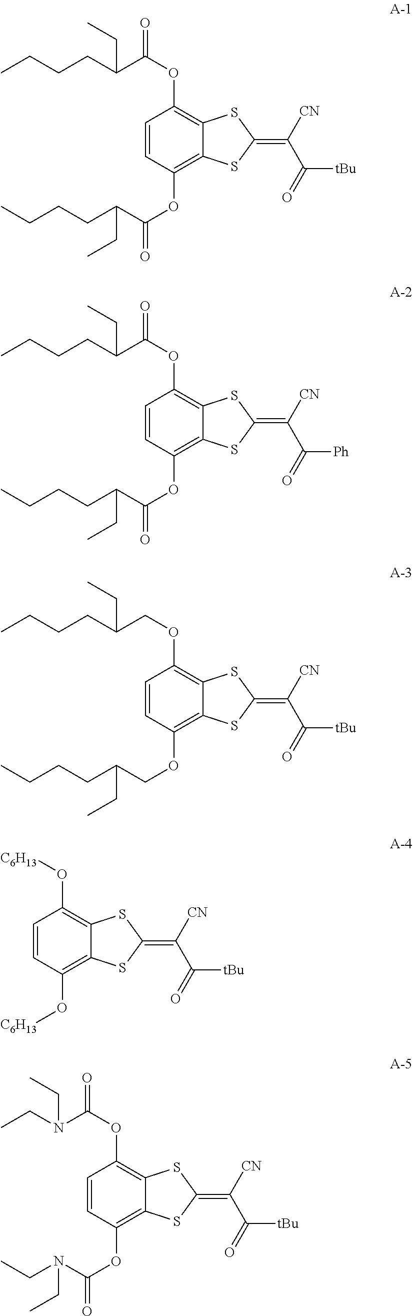

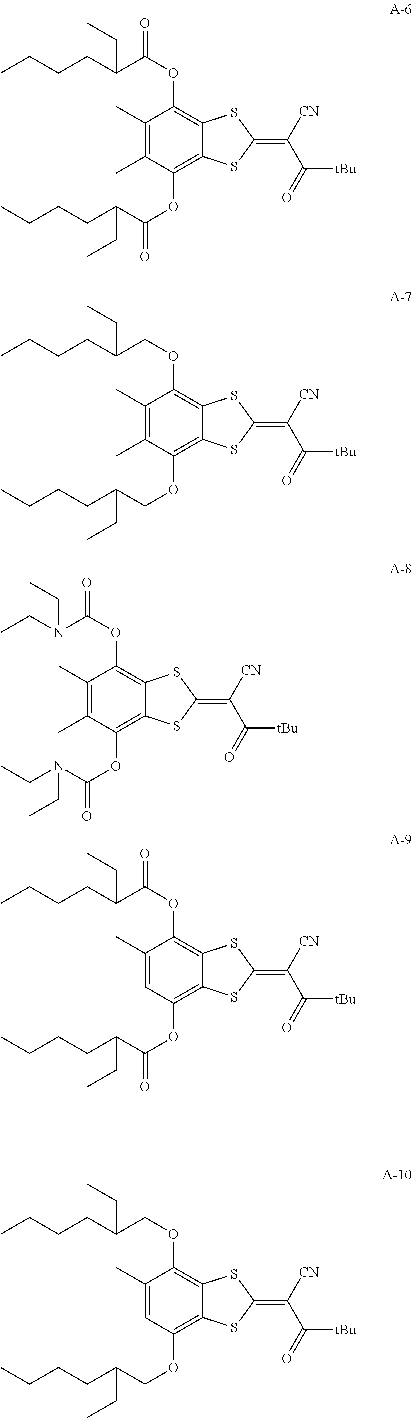

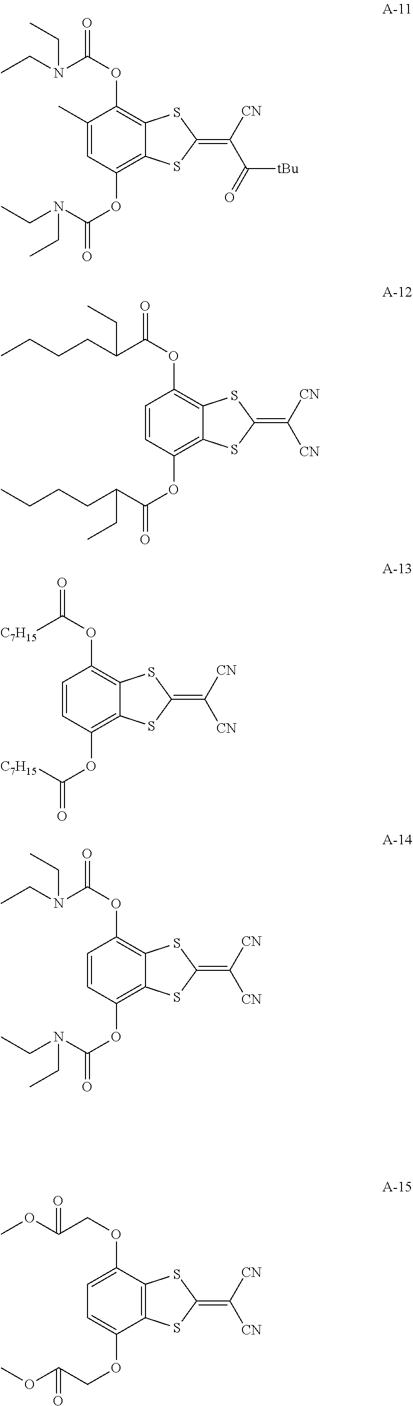

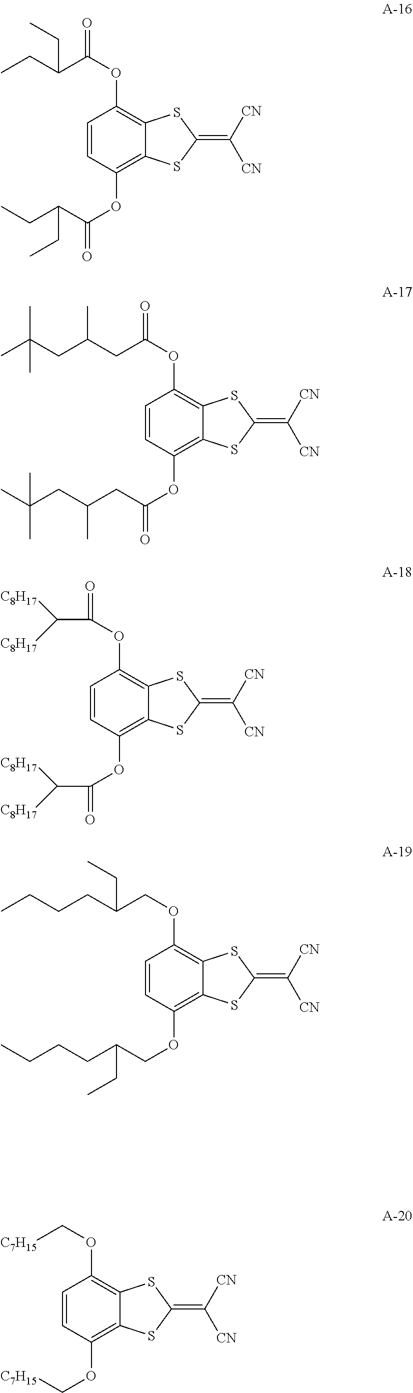

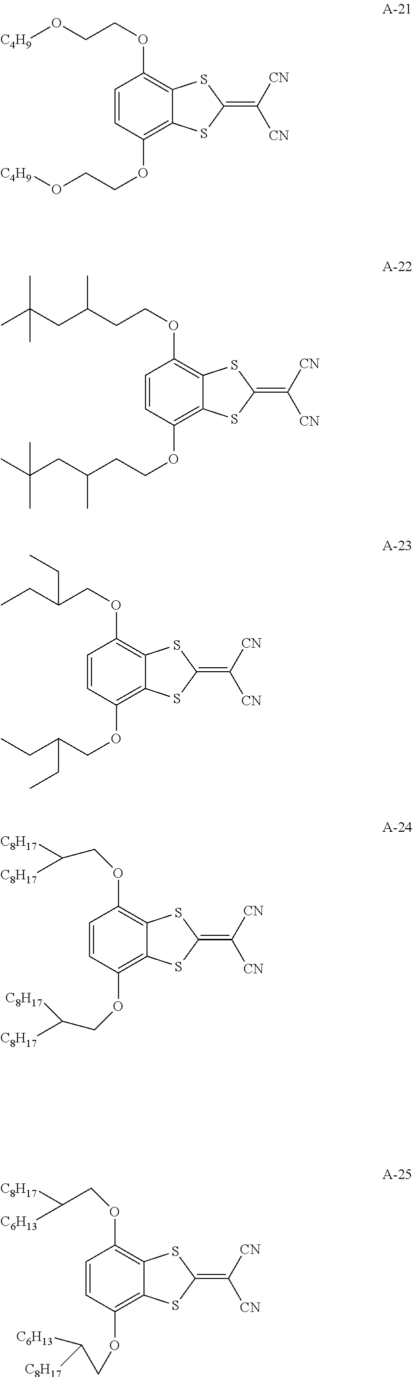

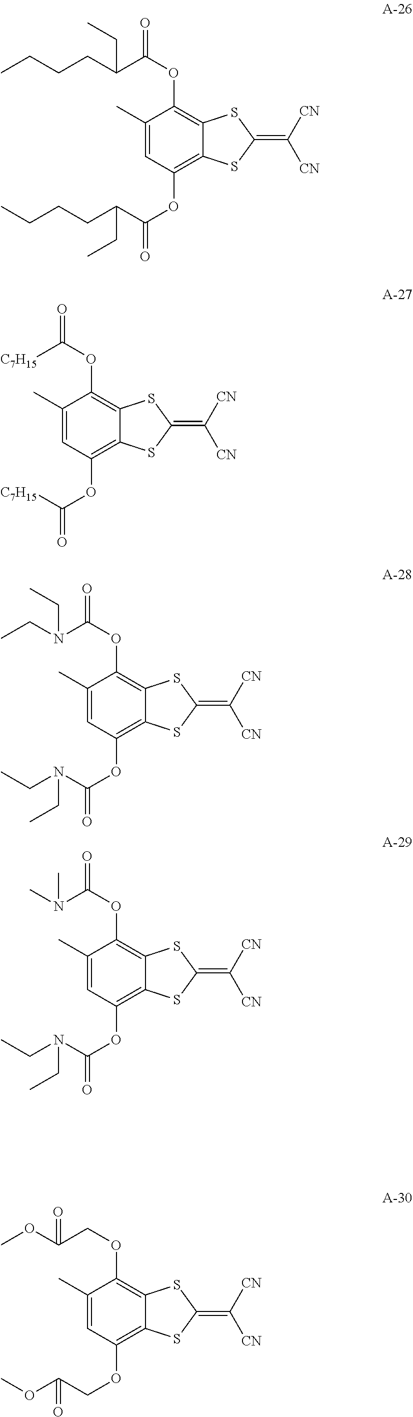

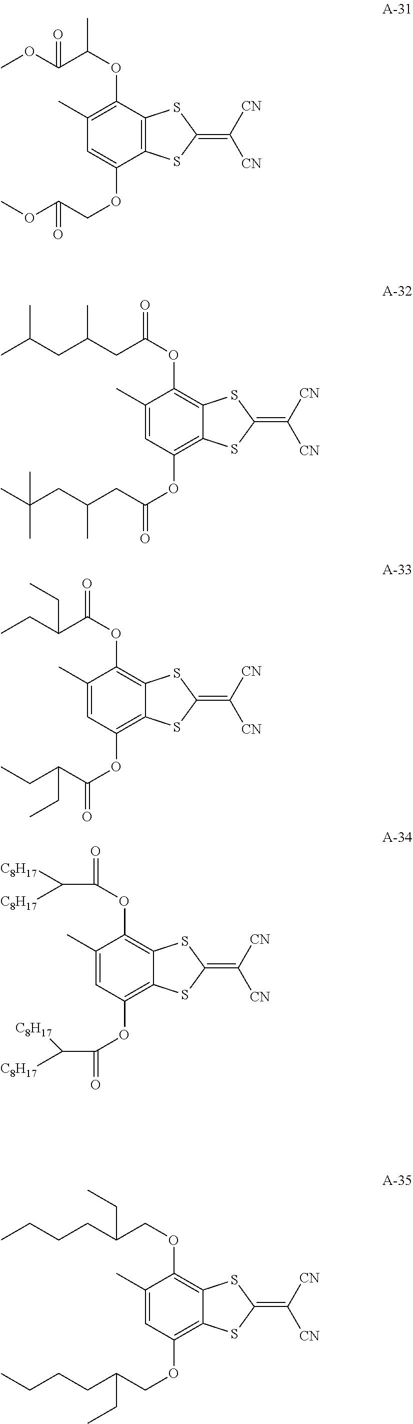

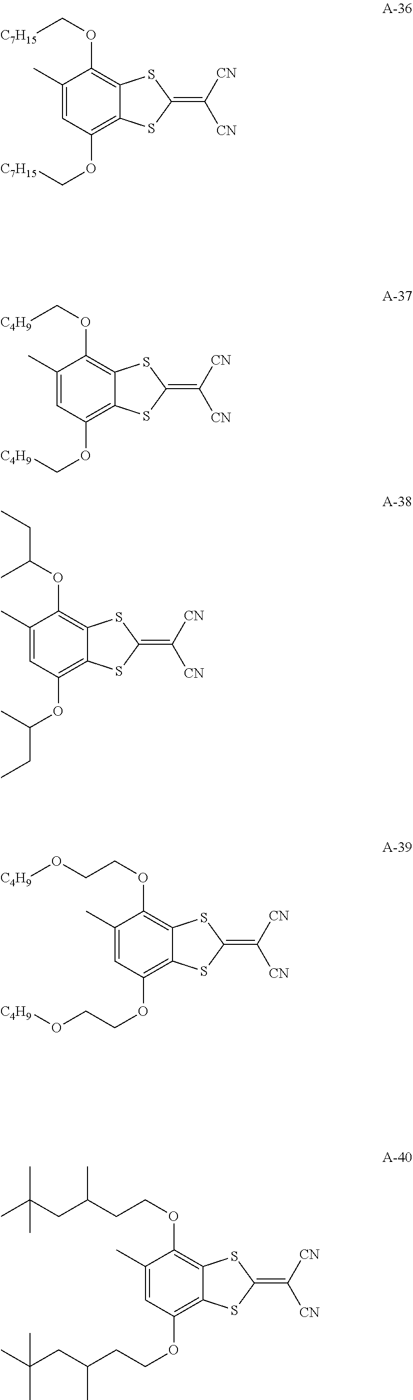

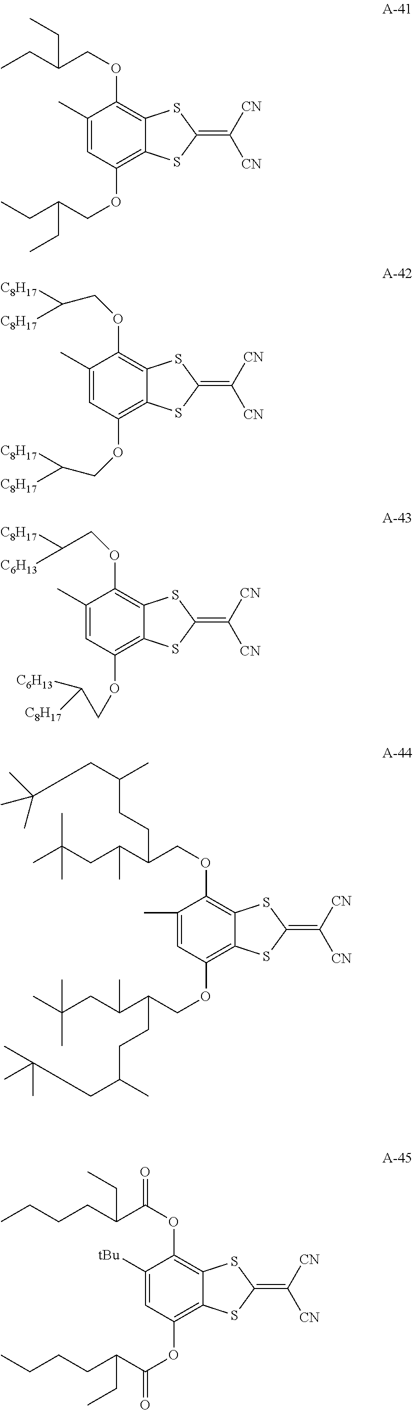

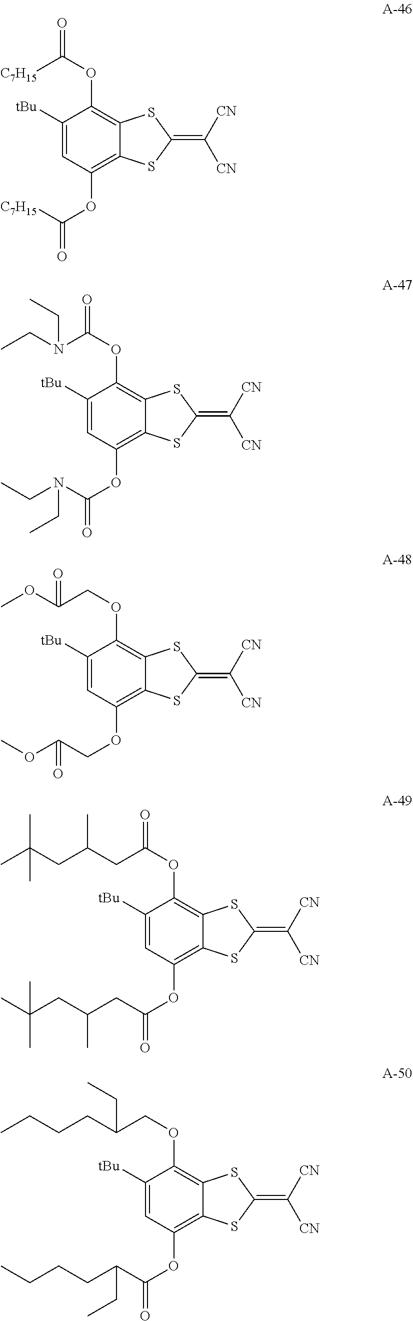

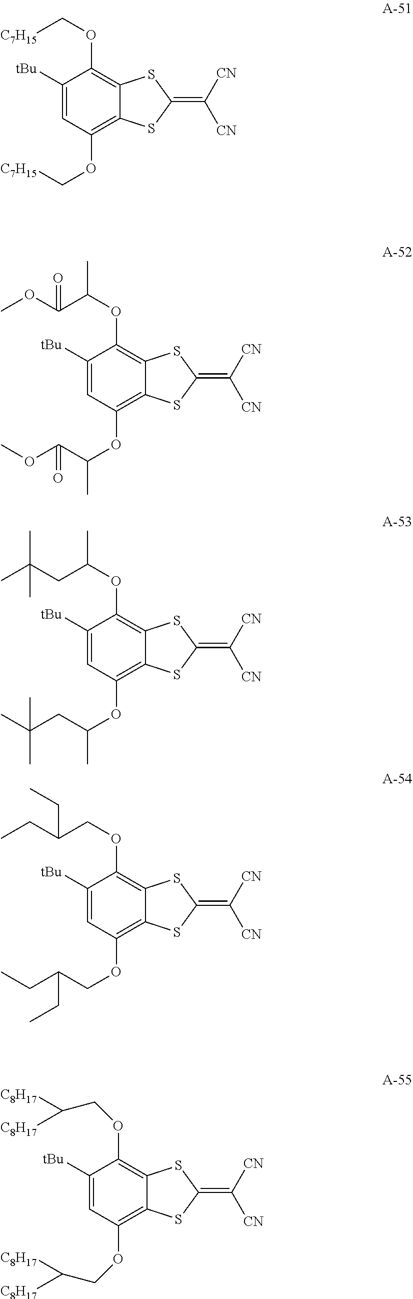

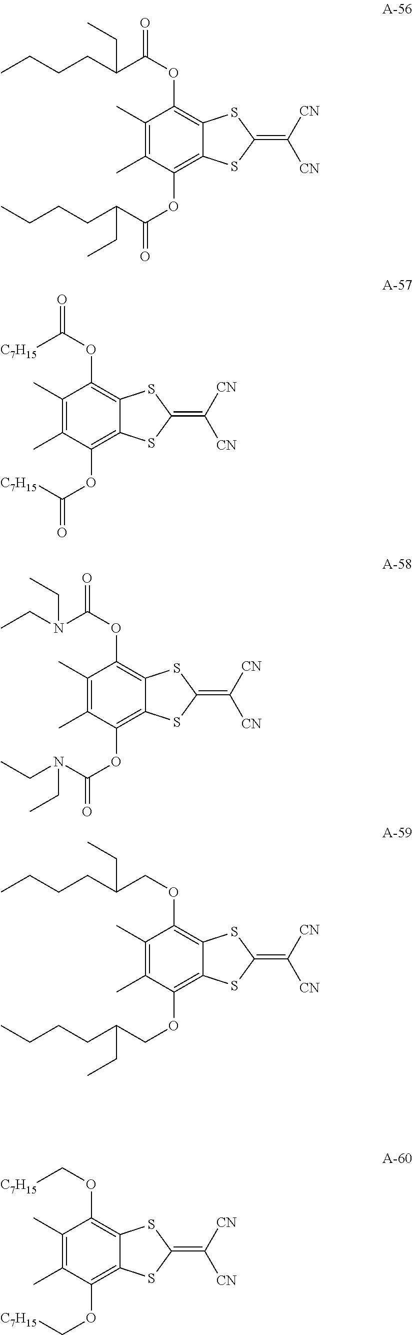

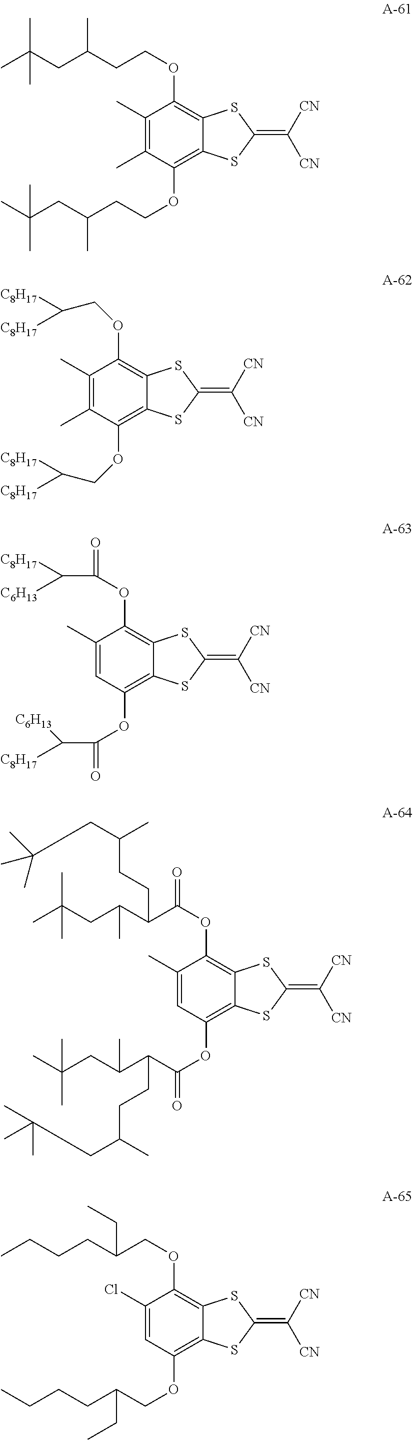

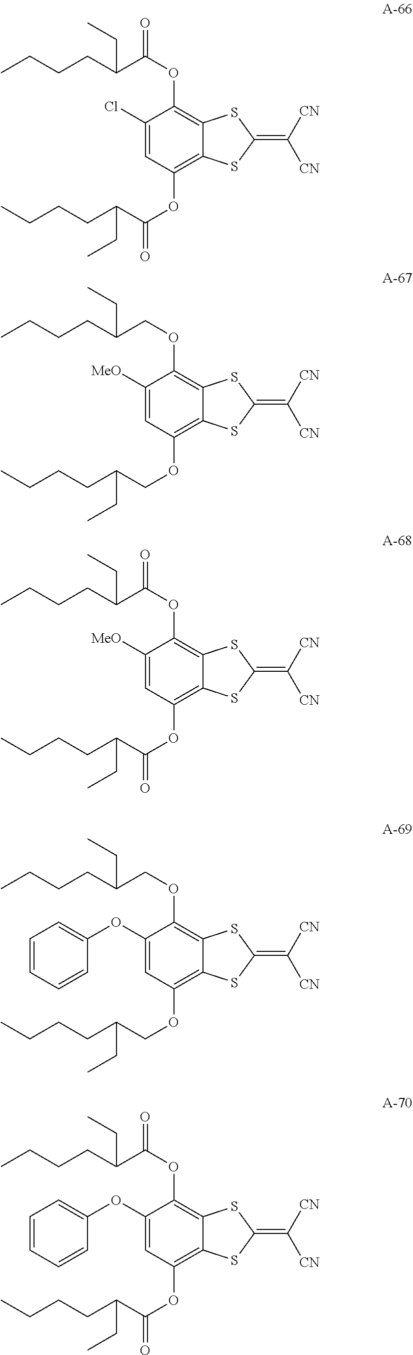

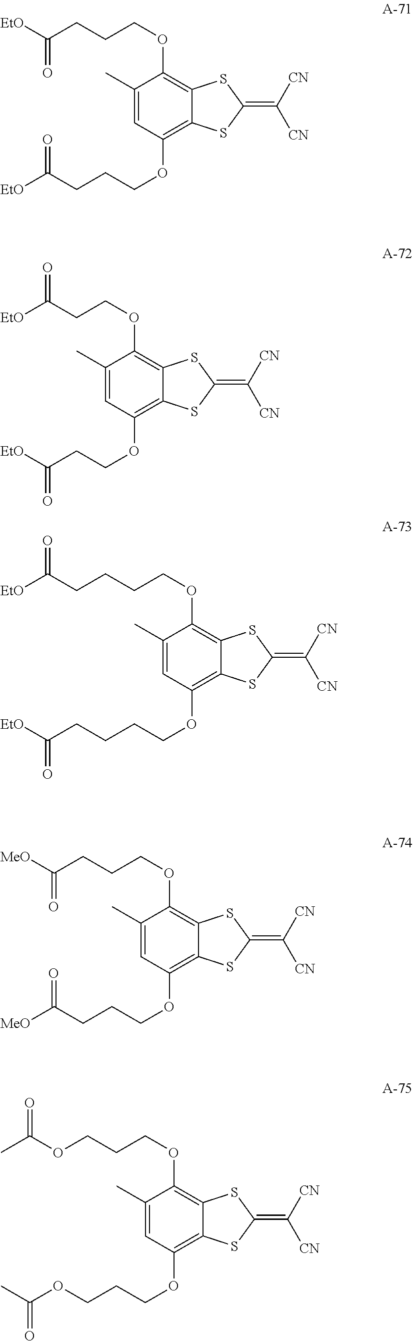

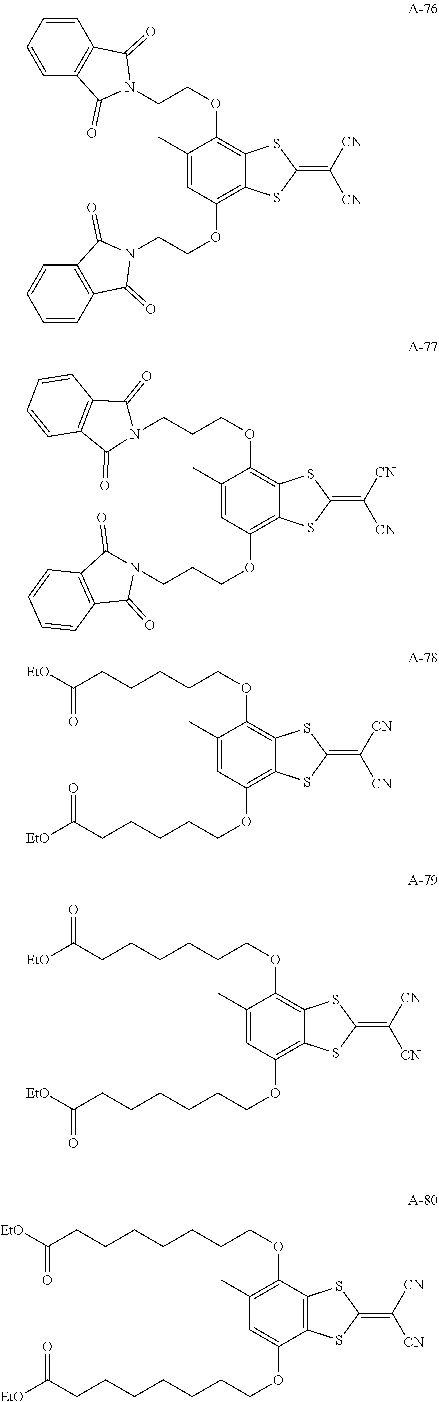

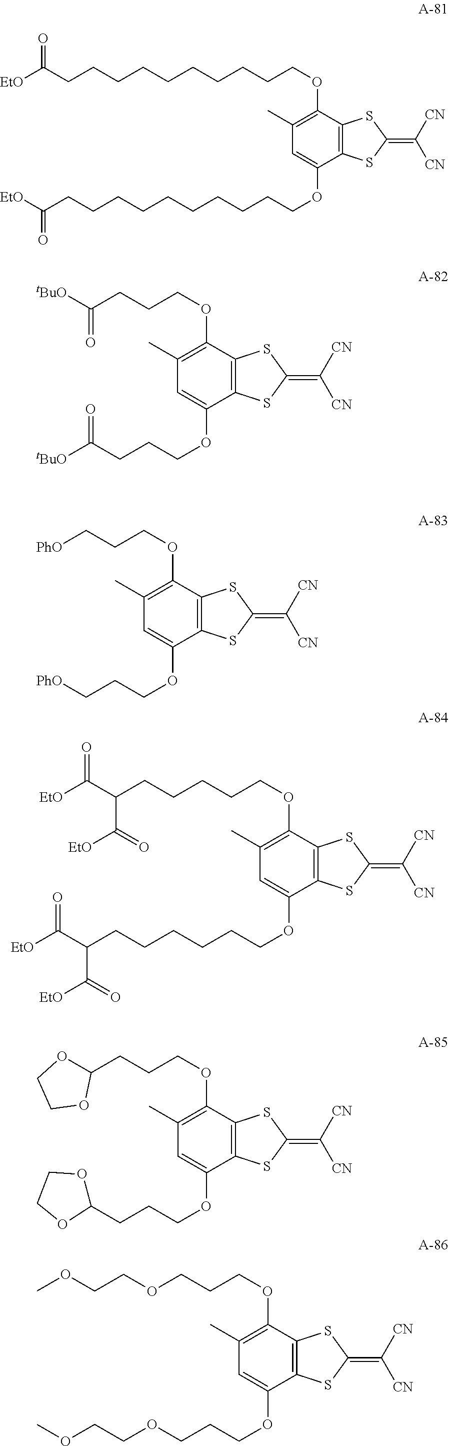 [SMILES: CCOC(=O)CCCCCCCCCCOc1cc(C)c(OCCCCCCCCCCC(=O)OCC)c2c1SC(=C(C#N)C#N)S2 ; Cc1cc(OCCCC(=O)OC(C)(C)C)c2c(c1OCCCC(=O)OC(C)(C)C)SC(=C(C#N)C#N)S2 ; Cc1cc(OCCCOc2ccccc2)c2c(c1OCCCOc1ccccc1)SC(=C(C#N)C#N)S2 ; CCOC(=O)C(CCCCCCOc1cc(C)c(OCCCCCC(C(=O)OCC)C(=O)OCC)c2c1SC(=C(C#N)C#N)S2)C(=O)OCC ; Cc1cc(OCCCC2OCCO2)c2c(c1OCCCC1OCCO1)SC(=C(C#N)C#N)S2 ; COCCOCCCOc1cc(C)c(OCCCOCCOC)c2c1SC(=C(C#N)C#N)S2]

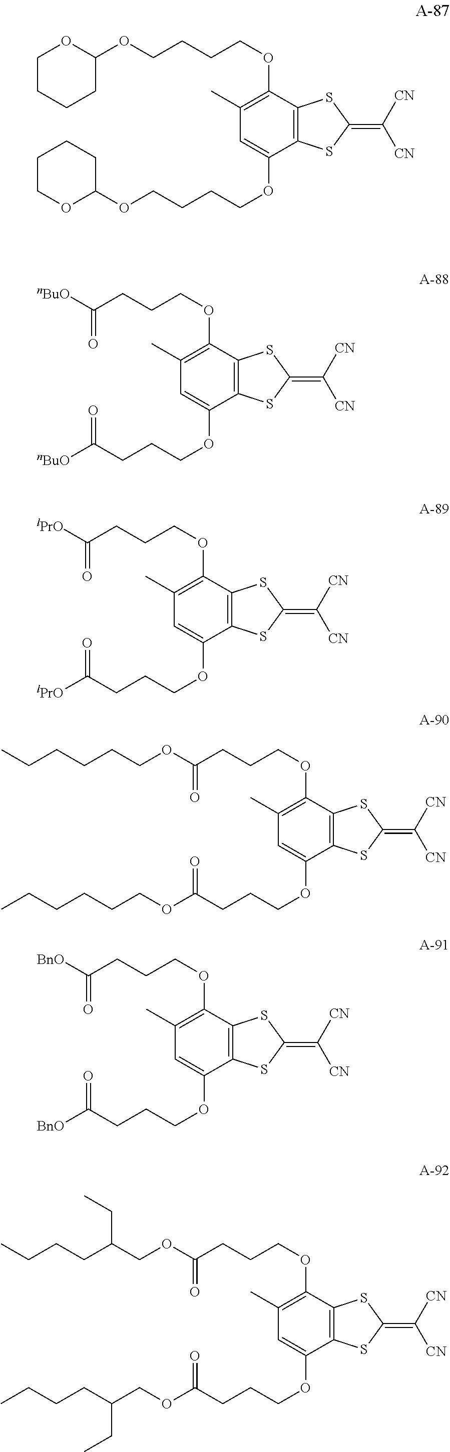

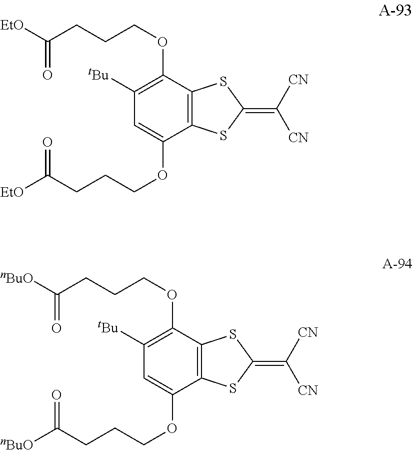

It is preferable that the ultraviolet absorbing agent according to the embodiment of the present invention is produced by reacting a compound represented by Formula (10) with a compound represented by Formula (20) to synthesize the compound (1) and treating the synthesized compound by bringing the compound into contact with an adsorbent.

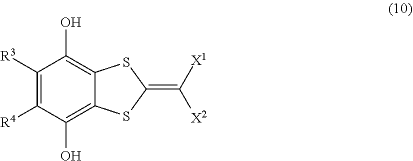

In Formula (10), X¹ and X² each independently represent a hydrogen atom or a substituent, and R³ and R⁴ each independently represent a hydrogen atom, a halogen atom, an alkyl group, an aryl group, an alkoxy group, or an aryloxy group, where at least one of X¹ or X² represents a substituent having a Hammett's substituent constant σp value of 0.2 or greater.

R²¹-E²¹ (20)

In Formula (20), E²¹ represents a group that reacts with a hydroxy group in Formula (10), and R²¹ represents an alkyl group, an acyl group, a carbamoyl group, an aryl group, an alkoxycarbonyl group, or an aryloxycarbonyl group.

X¹ and X² in Formula (10) each have the same definition as that for X¹ and X² in Formula (1). R³ and R⁴ in Formula (10) each have the same definition as that for R³ and R⁴ in Formula (1).

R²¹ in Formula (20) has the same definition as that for R¹ and R² in Formula (1). Examples of the group that is represented by E²¹ in Formula (20) and reacts with the hydroxy group of Formula (10) include —C(═O)Cl, —O(C═O)Cl, —NCO, —Cl, —Br, —I, —OSO₂D¹, and an oxiranyl group. D¹ represents a methyl group, an ethyl group, a phenyl group, or a 4-methylphenyl group.

The compound (1) can be synthesized with reference to the synthetic methods described in JP1974-011155B (JP-S49-011155B), JP2009-096971A, and the like.

Examples of the adsorbent used for the treatment performed on the compound (1) after synthesis include a resin-based adsorbent such as an ion exchange resin or a chelate resin, activated carbon, activated alumina, silica gel, zeolite, a hydrotalcite-like compound, and a mixed adsorbent (such as a mixture of zeolite and a ferrocyanide or a mixture of activated carbon and a ferrocyanide), and from the viewpoint of more effectively decreasing the absorbance at a wavelength of approximately 430 nm, activated carbon and activated alumina are preferable, and activated carbon is more preferable.

Examples of a method of treating the compound (1) after synthesis with an adsorbent include a method of stirring a liquid containing an adsorbent and the compound (1) after synthesis and a method of filling a column with an adsorbent and allowing a solution in which the compound (1) after synthesis is dissolved, to pass through the column. Among these, from the viewpoint of more effectively decreasing the absorbance at a wavelength of approximately 430 nm, a method of stirring a liquid containing an adsorbent and the compound (1) after synthesis is preferable. The treatment with the adsorbent may be repeated a plurality of times. The treatment time and the number of times of the treatment are not particularly limited. It is preferable that the treatment is performed such that the absorbance ratio is 0.01 or less.

The ultraviolet absorbing agent according to the embodiment of the present invention can be added to a resin and used as a resin composition. In addition, the ultraviolet absorbing agent can also be used as a solution by being dissolved in a solvent.

The ultraviolet absorbing agent according to the embodiment of the present invention can also be suitably used for applications in a case where the ultraviolet absorbing agent may be exposed to light including sunlight and ultraviolet rays. Specific examples include coating materials or films for window glass of houses, facilities, and transportation equipment; interior/exterior materials and interior/exterior paints of houses, facilities, and transportation equipment; members for light sources that emit ultraviolet rays, such as a fluorescent lamp and a mercury lamp; solar cells, precision machineries, electronic and electrical equipment, and members for a display device; containers or packaging materials for food, chemicals, and drugs; agricultural and industrial sheets; clothing textile products and fibers such as sportswear, stockings, and hats; lenses such as plastics lenses, contact lenses, glasses, and artificial eyes, or coating materials thereof; optical supplies such as optical filters, prisms, mirrors, and photographic materials; stationery such as tapes and inks; and marking boards, marking devices, and the surface coating materials thereof. For the details thereof, the description in paragraphs 0158 to 0218 of JP2009-263617A can be referred to, and the contents thereof are incorporated in the present specification.

<Resin Composition>

Hereinafter, a resin composition according to the embodiment of the present invention will be described in detail. The resin composition according to the embodiment of the present invention contains the above-described ultraviolet absorbing agent according to the embodiment of the present invention and a resin.

The content of the ultraviolet absorbing agent according to the embodiment of the present invention in the total solid content of the resin composition is preferably in a range of 0.01% to 50% by mass. The lower limit thereof is preferably 0.05% by mass or greater and more preferably 0.1% by mass or greater. The upper limit thereof is preferably 40% by mass or less, more preferably 30% by mass or less, and more preferably 20% by mass or less.

Further, the content of the compound (1) in the total solid content of the resin composition is preferably in a range of 0.01% to 50% by mass. The lower limit thereof is preferably 0.05% by mass or greater and more preferably 0.1% by mass or greater. The upper limit thereof is preferably 40% by mass or less, more preferably 30% by mass or less, and more preferably 20% by mass or less.

The resin composition may contain only one or two or more kinds of the compounds (1). In a case where the resin composition contains two or more kinds of the compounds (1), it is preferable that the total amount thereof is in the above-described range.

<<Resin>>

The resin composition according to the embodiment of the present invention contains a resin. The resin can be appropriately selected from resins that satisfy various physical properties, such as the transparency, the refractive index, and the workability, which are required depending on the applications or the purpose thereof.

Examples of the resin include a (meth)acrylic resin, an ene-thiol resin, a polyester resin, a polycarbonate resin, a vinyl polymer [such as a polydiene resin, a polyalkene resin, a polystyrene resin, a polyvinyl ether resin, a polyvinyl alcohol resin, a polyvinyl ketone resin, a polyfluorovinyl resin, or a polyvinyl bromide resin], a polythioether resin, a polyphenylene resin, a polyurethane resin, a polysulfonate resin, a nitroso polymer resin, a polysiloxane resin, a polysulfide resin, a polythioester resin, a polysulfone resin, a polysulfonamide resin, a polyamide resin, a polyimine resin, a polyurea resin, a polyphosphazene resin, a polysilane resin, a polysilazane resin, a polyfuran resin, a polybenzoxazole resin, a polyoxadiazole resin, a polybenzothiazinophenothiazine resin, a polybenzothiazole resin, a polypyrazinoquinoxaline resin, a polyquinoxaline resin, a polybenzoimidazoline resin, a polyoxoisoindoline resin, a polydioxoisoindoline resin, a polytriazine resin, a polypyridazine resin, a polypiperazine resin, a polypyridine resin, a polypiperidine resin, a polytriazole resin, a polypyrazole resin, a polypyrrolidine resin, a polycarborane resin, a polyoxabicyclononane resin, a polydibenzofuran resin, a polyphthalide resin, a polyacetal resin, a polyimide resin, a polyamide imide resin, an olefin resin, a cyclic olefin resin, an epoxy resin, and a cellulose acylate resin.

Examples of the (meth)acrylic resin include a polymer having a constitutional unit derived from (meth)acrylic acid and/or an ester thereof. Specific examples thereof include a polymer obtained by carrying out a polymerization reaction on at least one compound selected from the group consisting of (meth)acrylic acid, (meth)acrylic acid ester, (meth)acrylamide, and (meth)acrylonitrile.

Examples of the polyester resin include a polymer obtained by a reaction between a polyol (such as ethylene glycol, propylene glycol, glycerin, or trimethylolpropane) and a polybasic acid (for example, an aromatic dicarboxylic acid (such as terephthalic acid, isophthalic acid, naphthalenedicarboxylic acid, or a dicarboxylic acid in which a hydrogen atom of any of these aromatic rings is substituted with a methyl group, an ethyl group, a phenyl group, or the like), an aliphatic dicarboxylic acid having 2 to 20 carbon atoms (such as adipic acid, sebacic acid, or decanedicarboxylic acid) or an alicyclic dicarboxylic acid (such as cyclohexanedicarboxylic acid)), and a polymer (such as polycaprolactone) obtained by ring-opening polymerization of a cyclic ester compound such as a caprolactone monomer.

Examples of the epoxy resin include a bisphenol A type epoxy resin, a bisphenol F type epoxy resin, a phenol novolak type epoxy resin, a cresol novolak type epoxy resin, and an aliphatic epoxy resin. As the epoxy resin, a commercially available product on the market may be used, and examples of the commercially available product include the following products.

Examples of the commercially available product of the bisphenol A type epoxy resins include jER825, jER827, jER828, jER834, jER1001, jER1002, jER1003, jER1055, jER1007, jER1009, and jER1010 (all manufactured by Mitsubishi Chemical Corporation), and EPICLON860, EPICLON1050, EPICLON1051, and EPICLON1055 (all manufactured by DIC Corporation). Examples of the commercially available product of the bisphenol F type epoxy resins include jER806, jER807, jER4004, jER4005, jER4007, and jER4010 (all manufactured by Mitsubishi Chemical Corporation), EPICLON830 and EPICLON835 (both manufactured by DIC Corporation), and LCE-21 and RE-602S (both manufactured by Nippon Kayaku Co., Ltd.).

Examples of the commercially available product of the phenol novolak type epoxy resins include jER152, jER154, jER157S70, and jER157S65 (all manufactured by Mitsubishi Chemical Corporation), and EPICLON N-740, EPICLON N-770, and EPICLON N-775 (all manufactured by DIC Corporation). Examples of the commercially available product of the cresol novolak type epoxy resins include EPICLON N-660, EPICLON N-665, EPICLON N-670, EPICLON N-673, EPICLON N-680, EPICLON N-690, and EPICLON N-695 (all manufactured by DIC Corporation), and EOCN-1020 (manufactured by Nippon Kayaku Co., Ltd.). Examples of the commercially available product of the aliphatic epoxy resin include ADEKA RESIN EP Series (such as EP-40805, EP-40855, and EP-40885; manufactured by ADEKA Corporation), CELLOXIDE 2021P, CELLOXIDE 2081, CELLOXIDE 2083, CELLOXIDE 2085, EHPE3150, EPOLEAD PB 3600, and EPOLEAD PB 4700 (all manufactured by Daicel Corporation), DENACOL EX-212L, EX-214L, EX-216L, EX-321L, and EX-850L (all manufactured by Nagase ChemteX Corporation), ADEKA RESIN EP Series (such as EP-40005, EP-40035, EP-40105, and EP-40115; manufactured by ADEKA Corporation), NC-2000, NC-3000, NC-7300, XD-1000, EPPN-501, and EPPN-502 (all manufactured by ADEKA Co., Ltd.), and jER1031S (manufactured by Mitsubishi Chemical Corporation). Further, examples of the commercially available product of the epoxy resins include MARPROOF G-0150M, G-0105SA, G-0130SP, G-0250SP, G-1005S, G-1005SA, G-1010S, G-2050M, G-01100, and G-01758 (all manufactured by NOF Corporation, epoxy group-containing polymer).

As the cellulose acylate resin, the cellulose acylate described in paragraphs 0016 to 0021 of JP2012-215689A is preferably used. As the polyester resin, a commercially available product such as the VYLON Series (for example, VYLON 500, manufactured by Toyobo Co., Ltd.) can also be used. As a commercially available product of the (meth)acrylic resin, SK Dyne Series (for example, SK Dyne-SF2147, manufactured by Soken Chemical & Engineering Co., Ltd.) can also be used.

As the polystyrene resin, a resin having 50% by mass or greater of a repeating unit derived from a styrene-based monomer is preferable, a resin having 70% by mass or greater of a repeating unit derived from a styrene-based monomer is more preferable, and a resin having 85% by mass or greater of a repeating unit derived from a styrene-based monomer is still more preferable.

Specific examples of the styrene-based monomer include styrene and a derivative thereof. Here, the styrene derivative is a compound in which another group is bonded to styrene, and examples thereof include alkylstyrene such as o-methylstyrene, m-methylstyrene, p-methylstyrene, 2,4-dimethylstyrene, o-ethylstyrene, or p-ethylstyrene, and substituted styrene in which a hydroxyl group, an alkoxy group, a carboxyl group, or halogen is introduced to a benzene nucleus of styrene such as hydroxystyrene, tert-butoxystyrene, vinyl benzoic acid, o-chlorostyrene, or p-chlorostyrene.

In addition, the polystyrene resin may have a repeating unit derived from a monomer other than the styrene-based monomer. Examples of other monomers include alkyl (meth)acrylate such as methyl (meth)acrylate, cyclohexyl (meth)acrylate, methylphenyl (meth)acrylate, or isopropyl (meth)acrylate; an unsaturated carboxylic acid monomer such as methacrylic acid, acrylic acid, itaconic acid, maleic acid, fumaric acid, or cinnamic acid; an unsaturated dicarboxylic acid anhydride monomer which is an anhydride of maleic acid, itaconic acid, ethylmaleic acid, methylitaconic acid, or chloromaleic acid; an unsaturated nitrile monomer such as acrylonitrile or methacrylonitrile; and a conjugated diene such as 1,3-butadiene, 2-methyl-1,3-butadiene (isoprene), 2,3-dimethyl-1,3-butadiene, 1,3-pentadiene, or 1,3-hexadiene.

Examples of commercially available products of the polystyrene resin include AS-70 (acrylonitrile/styrene copolymer resin, manufactured by NIPPON STEEL Chemical & Material Co., Ltd.) and SMA2000P (styrene/maleic acid copolymer, manufactured by Kawahara Petrochemical Co., Ltd.).

The resin may contain an acid group. Examples of the acid group include a carboxyl group, a phosphoric acid group, a sulfonic acid group, and a phenolic hydroxy group. The acid group may be used alone or in combination of two or more kinds thereof. The resin containing an acid group can be used as an alkali-soluble resin or as a dispersant.

As the resin containing an acid group, the description in paragraphs 0558 to 0571 of JP2012-208494A (paragraphs 0685 to 0700 in the specification of US2012/0235099A) and the description in paragraphs 0076 to 0099 of JP2012-198408A can be referred to, and the contents thereof are incorporated in the present specification. Further, as the resin containing an acid group, ACRYBASE FF-426 (manufactured by Nippon Shokubai Co., Ltd.) can also be used.

The acid value of the resin containing an acid group is preferably in a range of 30 to 200 mgKOH/g. The lower limit of the acid value thereof is preferably 50 mgKOH/g or greater and more preferably 70 mgKOH/g or greater. Further, the upper limit of the acid value is preferably 150 mgKOH/g or less and more preferably 120 mgKOH/g or less. The acid value of the resin is a value calculated by performing measurement in conformity with JIS K 0070 (1992) and converting the measured value in terms of 1 mmol/g=56.1 mgKOH/g.

The resin may contain a curable group. Examples of the curable group include an ethylenically unsaturated bond-containing group, an epoxy group, a methylol group, and an alkoxysilyl group.

Examples of the ethylenically unsaturated bond-containing group include a vinyl group, a styrene group, an allyl group, a methallyl group, and a (meth)acryloyl group.

Examples of the alkoxysilyl group include a monoalkoxysilyl group, a dialkoxysilyl group, and a trialkoxysilyl group.

Examples of commercially available products of the resin containing a curable group include DIANAL BR Series (polymethyl methacrylate (PMMA), for example, DIANAL BR-80, BR-83, and BR-87; manufactured by Mitsubishi Chemical Corporation), Photomer 6173 (COOH-containing polyurethane acrylic oligomer, manufactured by Diamond Shamrock Co., Ltd.), VISCOAT R-264 and KS Resist 106 (both manufactured by Osaka Organic Chemical Industry Ltd.), CYCLOMER P Series (for example, ACA230AA) and PLACCEL CF200 Series (all manufactured by Daicel Corporation), Ebecryl 3800 (manufactured by Daicel UCB Co.), and Acrylic-RD-F8 (manufactured by Nippon Shokubai Co., Ltd.). In addition, examples thereof can also further include the commercially available products such as the products described in the section of the epoxy resin above.

For example, in a case where the resin composition according to the embodiment of the present invention is used for a lens (for example, a spectacle lens), suitable examples of the resin include a thermoplastic resin such as a carbonate resin or a (meth)acrylic resin (such as polymethyl methacrylate (PMMA)), and a thermosetting resin such as a urethane resin. Examples of commercially available products of the carbonate resins on the market include a polycarbonate resin composition (trade name: CALIBRE 200-13, manufactured by Sumitomo Dow Ltd.) and a diethylene glycol bisallyl carbonate resin (trade name: CR-39, manufactured by PPG Industries). As the urethane resin, a thiourethane resin is preferable. Examples of commercially available products of the thiourethane resin on the market include thiourethane resin monomers (trade names: MR-7, MR-8, MR-10, and MR-174: all trade names; manufactured by Mitsui Chemicals, Inc.).

In addition, a pressure sensitive adhesive or an adhesive can also be used for the resin. Examples of the pressure sensitive adhesive include an acrylic pressure sensitive adhesive, a rubber-based pressure sensitive adhesive, and a silicone-based pressure sensitive adhesive. The acrylic pressure sensitive adhesive denotes a pressure sensitive adhesive containing a polymer of a (meth)acrylic monomer ((meth)acrylic polymer). Examples of the adhesive include a urethane resin adhesive, a polyester adhesive, an acrylic resin adhesive, an ethylene vinyl acetate resin adhesive, a polyvinyl alcohol adhesive, a polyamide adhesive, and a silicone adhesive. Among these, from the viewpoint of excellent adhesive strength, a urethane resin adhesive or a silicone adhesive is preferable as the adhesive. As the adhesive, a commercially available product on the market may be used, and examples of the commercially available product thereof include a urethane resin adhesive (LIS-073-50U: trade name, manufactured by of Toyo Ink Co., Ltd.) and an acrylic pressure sensitive adhesive (SK Dyne-SF2147: trade name, manufactured by Soken Chemical & Engineering Co., Ltd.).

The weight-average molecular weight (Mw) of the resin is preferably in a range of 2,000 to 2,000,000. The lower limit of Mw of the resin is more preferably 5,000 or greater, still more preferably 10,000 or greater, and particularly preferably 50,000 or greater. The upper limit of Mw of the resin is more preferably 1,000,000 or less, still more preferably 500,000 or less, and particularly preferably 200,000 or less. Further, in a case where an epoxy resin is used, the weight-average molecular weight (Mw) of the epoxy resin is preferably 100 or greater and more preferably in a range of 200 to 2,000,000. The upper limit of Mw of the epoxy resin is still more preferably 1,000,000 or less and particularly preferably 500,000 or less. The lower limit of Mw of the epoxy resin is more preferably 2,000 or greater.

The weight-average molecular weight (Mw) is a value measured by gel permeation chromatography (GPC). The measurement according to GPC is performed using HLC (registered trademark)-8020GPC (manufactured by Tosoh Corporation) as a measuring device, three columns of TSKgel (registered trademark) Super Multipore HZ-H (manufactured by Tosoh Corporation, 4.6 mmID×15 cm), and tetrahydrofuran (THF) as an eluent. Further, the measurement is performed under measurement conditions of a sample concentration of 0.45% by mass, a flow rate of 0.35 ml/min, a sample injection volume of 10 μl, and a measurement temperature of 40° C. using an RI detector. Further, the calibration curve is prepared using eight samples of “F-40”, “F-20”, “F-4”, “F-1”, “A-5000”, “A-2500”, “A-1000”, and “n-propylbenzene” which are “Standard Samples TSK standard, polystyrene” (manufactured by Tosoh Corporation).

The total light transmittance of the resin is preferably 80% or greater, more preferably 85% or greater, and still more preferably 90% or greater. In the present specification, the total light transmittance of the resin is a value measured based on the contents described in “The Fourth Series of Experimental Chemistry 29 Polymer Material” (Maruzen, 1992), pp. 225 to 232, edited by the Chemical Society of Japan.

The content of the resin in the total solid content of the resin composition is preferably in a range of 1% to 99.9% by mass. The lower limit thereof is preferably 30% by mass or greater, more preferably 50% by mass or greater, and still more preferably 70% by mass or greater. The upper limit thereof is preferably 95% by mass or less, more preferably 90% by mass or less, and still more preferably 80% by mass or less. The resin composition may contain only one or two or more kinds of resins. In a case where the polymerization composition contains two or more kinds of resins, it is preferable that the total amount thereof is in the above-described range.

<<Other Ultraviolet Absorbing Agents>>

The resin composition according to the embodiment of the present invention can contain other ultraviolet absorbing agents in addition to the above-described ultraviolet absorbing agent according to the embodiment of the present invention (hereinafter, also referred to as other ultraviolet absorbing agents). According to this aspect, a cured substance capable of shielding light having a wavelength in the ultraviolet region over a wide range can be formed.

The maximum absorption wavelengths of the other ultraviolet absorbing agents are present preferably in a wavelength range of 300 to 380 nm, more preferably in a wavelength range of 300 to 370 nm, still more preferably in a wavelength range of 310 to 360 nm, and particularly preferable in a wavelength range of 310 to 350 nm.

Examples of the other ultraviolet absorbing agents include an aminobutadiene-based ultraviolet absorbing agent, a dibenzoylmethane-based ultraviolet absorbing agent, a benzotriazole-based ultraviolet absorbing agent, a benzophenone-based ultraviolet absorbing agent, a salicylic acid-based ultraviolet absorbing agent, an acrylate-based ultraviolet absorbing agent, and a triazine-based ultraviolet absorbing agent. Among these, a benzotriazole-based ultraviolet absorbing agent, a benzophenone-based ultraviolet absorbing agent, and a triazine-based ultraviolet absorbing agent are preferable, and a benzotriazole-based ultraviolet absorbing agent and a triazine-based ultraviolet absorbing agents are more preferable. Specific examples of the other ultraviolet absorbing agents include compounds described in paragraphs 0065 to 0070 of JP2009-263616A and compounds described in paragraph 0065 of WO2017/122503A, and the contents thereof are incorporated in the present specification. Preferred examples of the other ultraviolet absorbing agents include 2-(2′-hydroxy-5′-t-butylphenyl)benzotriazole, 2-(2′-hydroxy-3′-t-butyl-5′-methylphenyl)-5-chlorobenzotriazole, 2-(4-butoxy-2-hydroxyphenyl)-4,6-di(4-butoxyphenyl)-1,3,5-triazine, 2-[2-hydroxy-4-(2-hydroxy-3-butyloxypropoxy)phenyl]-4,6-bis(2,4-dimethyl)-1,3,5-triazine, 2,2′,4,4′-tetrahydroxybenzophenone, and 2,2′-dihydroxy-4,4′-dimethoxybenzophenone.

In a case where the resin composition contains other ultraviolet absorbing agents, the content of the other ultraviolet absorbing agents in the total solid content of the resin composition is preferably in a range of 0.01% to 50% by mass. The lower limit thereof is preferably 0.05% by mass or greater and more preferably 0.1% by mass or greater. The upper limit thereof is preferably 40% by mass or less, more preferably 30% by mass or less, and more preferably 20% by mass or less.

Further, the total content of the compound (1) and other ultraviolet absorbing agents in the total solid content of the resin composition is preferably in a range of 0.01% to 50% by mass. The lower limit thereof is preferably 0.05% by mass or greater and more preferably 0.1% by mass or greater. The upper limit thereof is preferably 40% by mass or less, more preferably 30% by mass or less, and more preferably 20% by mass or less.

The resin composition may contain only one or two or more kinds of other ultraviolet absorbing agents. In a case where the resin composition contains two or more kinds of other ultraviolet absorbing agents, it is preferable that the total amount thereof is in the above-described range.

<<Polymerizable Compound>>

The resin composition according to the embodiment of the present invention can contain a polymerizable compound. As the polymerizable compound, a compound that can be polymerized and cured by applying energy can be used without limitation. Examples of the polymerizable compound include a compound containing an ethylenically unsaturated bond-containing group. Examples of the ethylenically unsaturated bond-containing group include a vinyl group, a styrene group, an allyl group, a methallyl group, and a (meth)acryloyl group.

The polymerizable compound may be, for example, any one of a monomer, a prepolymer (that is, a dimer, a trimer, or an oligomer), and a mixture thereof, and a (co)polymer of a compound selected from the monomer and the prepolymer.

Examples of the polymerizable compound include an unsaturated carboxylic acid (such as acrylic acid, methacrylic acid, itaconic acid, crotonic acid, isocrotonic acid, or maleic acid), an ester of an unsaturated carboxylic acid, an amide of an unsaturated carboxylic acid, and a (co)polymer of the unsaturated carboxylic acid, the ester thereof, or the amide thereof. Among these, esters of an unsaturated carboxylic acid and an aliphatic polyhydric alcohol, amides of an unsaturated carboxylic acid and an aliphatic polyvalent amine, and homopolymers or copolymers thereof are preferable.

Further, examples of the polymerizable compound include an addition reactant of an unsaturated carboxylic acid ester or an unsaturated carboxylic acid amide containing a nucleophilic substituent (such as a hydroxy group, an amino group, or a mercapto group) and a monofunctional or polyfunctional isocyanate compound or an epoxy compound; a dehydration condensation reactant of an unsaturated carboxylic acid ester or an unsaturated carboxylic acid amide containing a nucleophilic substituent and a monofunctional or polyfunctional carboxylic acid; an addition reactant of an unsaturated carboxylic acid ester or an unsaturated carboxylic acid amide containing an electrophilic substituent (such as an isocyanate group or an epoxy group), a monofunctional or polyfunctional alcohol, and an amine or thiol; and a substitution reactant of an unsaturated carboxylic acid ester or an unsaturated carboxylic acid amide containing a releasable substituent (such as a halogen group or a tosyloxy group), a monofunctional or polyfunctional alcohol, and an amine or thiol. Further, a compound obtained by substituting the above-described unsaturated carboxylic acid with an unsaturated phosphonic acid, styrene, vinyl ether, or the like can also be used.

Further, a plurality of compounds with different numbers of functional groups or a plurality of compounds with different kinds of polymerizable groups (for example, acrylic acid ester, methacrylic acid ester, a styrene compound, or a vinyl ether compound) may be used in combination as the polymerizable compound.

Examples of commercially available products of the polymerizable compound include KYARAD (registered trademark) Series (for example, PET-30 and TPA-330, manufactured by Nippon Kayaku Co., Ltd.), POLYVEST (registered trademark) 110M and the like (manufactured by Evonik Industries AG), and a polyfunctional (meth)acrylate compound of NK Ester Series (for example, NK Ester A-9300, manufactured by Shin Nakamura Chemical Industry Co., Ltd.).

In a case where the resin composition contains a polymerizable compound, the content of the polymerizable compound in the total solid content of the resin composition is preferably in a range of 0.1% to 90% by mass. The lower limit thereof is preferably 1% by mass or greater and more preferably 5% by mass or greater. The upper limit thereof is preferably 80% by mass or less and more preferably 70% by mass or less. The resin composition may contain only one or two or more kinds of polymerizable compounds. In a case where the polymerizable composition contains two or more kinds of polymerizable compounds, it is preferable that the total amount thereof is in the above-described ranges.

<<Polymerization Initiator>>

The resin composition can contain a polymerization initiator. As the polymerization initiator, a compound capable of generating an initiating species required for the polymerization reaction by applying energy can be used. The polymerization initiator can be appropriately selected from, for example, a photopolymerization initiator and a thermal polymerization initiator, and a photopolymerization initiator is preferable.

For example, a photopolymerization initiator having photosensitivity to light rays from an ultraviolet region to a visible region is preferable as the photopolymerization initiator. Further, the photopolymerization initiator may be an activator that causes some action with a photoexcited sensitizer to generate an active radical.

Examples of the photoradical polymerization initiator include a halogenated hydrocarbon derivative (such as a compound having a triazine skeleton or a compound having an oxadiazole skeleton), an acylphosphine compound, hexaarylbiimidazole, an oxime compound, an organic peroxide, a thio compound, a ketone compound, an aromatic onium salt, an aminoacetophenone compound, and a hydroxyacetophenone compound. Examples of the aminoacetophenone compound include aminoacetophenone-based initiators described in JP2009-191179A and JP1998-291969A (JP-H10-291969A). Examples of the acylphosphine compound include the acylphosphine-based initiator described in JP4225898B. Examples of the oxime compound include the compounds described in JP2001-233842A, the compounds described in JP2000-080068A, the compounds described in JP2006-342166A, and the compounds described in paragraphs 0073 to 0075 of JP2016-006475A. Among the examples of the oxime compound, an oxime ester compound is preferable. As the photoradical polymerization initiator, a synthetic product may be used, or a commercially available product on the market may be used.

Examples of commercially available products of the hydroxyacetophenone compound include Omnirad 184, Omnirad 1173, Omnirad 2959, and Omnirad 127 (all manufactured by IGM Resins B. V.). Examples of commercially available products of the aminoacetophenone compound include Omnirad 907, Omnirad 369, Omnirad 369E, and Omnirad 379EG (all manufactured by IGM Resins B. V). Examples of commercially available products of the acylphosphine compound include Omnirad 819 and Omnirad TPO (both manufactured by IGM Resins B. V.). Examples of commercially available products of the oxime compound include Irgacure OXE01, Irgacure OXE02 (manufactured by BASF SE), and Irgacure OXE03 (manufactured by BASF SE).

The thermal radical polymerization initiator is not particularly limited, and a known thermal radical polymerization initiator can be used. Examples thereof include an azo-based compound such as dimethyl 2,2′-azobis(isobutyrate), 2,2′-azobisisobutyronitrile, 2,2′-azobis(2,4-dimethyl-4-methoxyvaleronitrile), 2,2′-azobis(2,4-dimethylvaleronitrile), dimethyl-2,2′-azobis(2-methylpropionate), 2,2′-azobis(2-methylbutyronitrile), 1,1′-azobis(cyclohexane-1-carbonitrile), 2,2′-azobis(N-butyl-2-methylpropionamide), dimethyl-1,1′-azobis(1-cyclohexanecarboxylate), or 2,2′-azobis[2-(2-imidazolin-2-yl)propane]dihydrochloride; an organic peroxide such as 1,1-di(t-hexylperoxy)cyclohexane, 1,1-di(t-butylperoxy)cyclohexane, 2,2-di(4,4-di-(t-butylperoxy)cyclohexyl)propane, t-hexyl peroxy isopropyl monocarbonate, t-butyl peroxy-3,5,5-trimethyl hexanoate, t-butyl peroxy laurate, dicumyl peroxide, di-t-butyl peroxide, t-butyl peroxy-2-ethyl hexanoate, t-hexyl peroxy-2-ethyl hexanoate, cumene hydroperoxide, or t-butyl hydroperoxide; and an inorganic peroxide such as potassium persulfate, ammonium persulfate, or hydrogen peroxide.

In a case where the resin composition contains a polymerization initiator, the content of the polymerization initiator in the total solid content of the resin composition is preferably in a range of 0.1% to 20% by mass. The lower limit thereof is preferably 0.3% by mass or greater and more preferably 0.4% by mass or greater. The upper limit thereof is preferably 15% by mass or less and more preferably 10% by mass or less. The resin composition may contain only one or two or more kinds of polymerization initiators. In a case where the polymerization composition contains two or more kinds of polymerization initiators, it is preferable that the total amount thereof is in the above-described range.

<<Acid Generator>>

The resin composition according to the embodiment of the present invention can contain an acid generator. The acid generator may be a photoacid generator or a thermal acid generator. In the present specification, an acid generator denotes a compound which generates an acid by applying energy such as heat or light. Further, the thermal acid generator denotes a compound that generates an acid by thermal decomposition. Further, the photoacid generator denotes a compound that generates an acid by light irradiation. Examples of the kind of acid generator, specific compounds, and preferred examples thereof include the compounds described in paragraphs 0066 to 0122 of JP2008-013646A, and these compounds can also be applied to the present invention.

As the thermal acid generator, a compound having a thermal decomposition temperature of 130° C. to 250° C. is preferable, and a compound having a thermal decomposition temperature of 150° C. to 220° C. is more preferable. Examples of the thermal acid generator include compounds that generate low nucleophilic acids such as a sulfonic acid, a carboxylic acid, and disulfonylimide by heating. As the acid generated by the thermal acid generator, an acid having a pKa of 4 or less is preferable, an acid having a pKa of 3 or less is more preferable, and an acid having a pKa of 2 or less is still more preferable. For example, a sulfonic acid, an alkylcarboxylic acid substituted with an electron withdrawing group, an arylcarboxylic acid, or disulfonylimide is preferable. Examples of the electron withdrawing group include a halogen atom such as a fluorine atom, a haloalkyl group such as a trifluoromethyl group, a nitro group, and a cyano group.

Examples of the photoacid generator include an onium salt compound such as a diazonium salt, a phosphonium salt, a sulfonium salt, or an iodonium salt, which are decomposed by light irradiation to generate an acid, and a sulfonate compound such as imide sulfonate, oxime sulfonate, diazodisulfone, disulfone, or ortho-nitrobenzyl sulfonate. Examples of commercially available products of the photoacid generator include WPAG-469 (manufactured by FUJIFILM Wako Pure Chemical Corporation), CPI-100P (manufactured by San-Apro Ltd.), and Irgacure 290 (manufactured by BASF SE). Further, 2-isopropylthioxanthone or the like can also be used as the photoacid generator.

In a case where the resin composition contains an acid generator, the content of the acid generator is preferably in a range of 0.1 to 100 parts by mass, more preferably in a range of 0.1 to 50 parts by mass, and still more preferably in a range of 0.1 to 20 parts by mass with respect to 100 parts by mass of the resin. The resin composition may contain only one or two or more kinds of acid generators. In a case where the resin composition contains two or more kinds of acid generators, it is preferable that the total amount thereof is in the above-described range.

<<Catalyst>>

The resin composition can contain a catalyst. Examples of the catalyst include an acid catalyst such as hydrochloric acid, sulfuric acid, acetic acid, or propionic acid and a base catalyst such as sodium hydroxide, potassium hydroxide, or triethylamine. In a case where the resin composition contains a catalyst, the content of the catalyst is preferably in a range of 0.1 to 100 parts by mass, more preferably in a range of 0.1 to 50 parts by mass, and still more preferably in a range of 0.1 to 20 parts by mass with respect to 100 parts by mass of the resin. The resin composition may contain only one or two or more kinds of catalysts. In a case where the resin composition contains two or more kinds of catalysts, it is preferable that the total amount thereof is in the above-described range.

<<Silane Coupling Agent>>

The resin composition according to the embodiment of the present invention may contain a silane coupling agent. According to this aspect, the adhesiveness of the film to be obtained to the support can be further improved. In the present invention, the silane coupling agent denotes a silane compound containing a hydrolyzable group and other functional groups. Further, the hydrolyzable group denotes a substituent that is directly bonded to a silicon atom and can form a siloxane bond by at least one of a hydrolysis reaction or a condensation reaction. Examples of the hydrolyzable group include a halogen atom, an alkoxy group, and an acyloxy group. Among these, an alkoxy group is preferable. That is, it is preferable that the silane coupling agent is a compound containing an alkoxysilyl group. Examples of the functional group other than the hydrolyzable group include a vinyl group, a (meth)allyl group, a (meth)acryloyl group, a mercapto group, an epoxy group, an oxetanyl group, an amino group, a ureido group, a sulfide group, and an isocyanate group, and a phenyl group. Among these, an amino group, a (meth)acryloyl group, and an epoxy group are preferable. Specific examples of the silane coupling agent include the compounds described in paragraphs 0018 to 0036 of JP2009-288703A and the compounds described in paragraphs 0056 to 0066 of JP2009-242604A, and the contents thereof are incorporated in the present specification. Examples of commercially available products of the silane coupling agent include A-50 (organosilane) (manufactured by Soken Chemical & Engineering Co., Ltd.). The content of the silane coupling agent in the total solid content of the resin composition is preferably in a range of 0.1% to 5% by mass. The upper limit thereof is preferably 3% by mass or less and more preferably 2% by mass or less. The lower limit thereof is preferably 0.5% by mass or greater and more preferably 1% by mass or greater. The silane coupling agent may be used alone or in combination of two or more kinds thereof. In a case where two or more kinds of silane coupling agents are used, it is preferable that the total amount is in the above-described range.

<<Surfactant>>

The resin composition according to the embodiment of the present invention can contain a surfactant. Examples of the surfactant include the surfactants described in paragraph 0017 of JP4502784B and paragraphs 0060 to 0071 of JP2009-237362A.

As the surfactant, a nonionic surfactant, a fluorine-based surfactant, or a silicone-based surfactant is preferable.

Examples of commercially available products of the fluorine-based surfactant include MEGAFACE F-171, F-172, F-173, F-176, F-177, F-141, F-142, F-143, F-144, F-437, F-475, F-477, F-479, F-482, F-551-A, F-552, F-554, F-555-A, F-556, F-557, F-558, F-559, F-560, F-561, F-565, F-563, F-568, F-575, F-780, EXP, MFS-330, R-41, R-41-LM, R-01, R-40, R-40-LM, RS-43, TF-1956, RS-90, R-94, RS-72-K, and DS-21 (all manufactured by DIC Corporation), FLUORARD FC430, FC431, and FC171 (all manufactured by Sumitomo 3M Ltd.), SURFLON S-382, SC-101, SC-103, SC-104, SC-105, SC-1068, SC-381, SC-383, S-393, and KH-40 (all manufactured by AGC Inc.), PolyFox PF636, PF656, PF6320, PF6520, and PF7002 (all manufactured by OMNOVA Solutions Inc.), and FTERGENT 710FM, 610FM, 601AD, 601ADH2, 602A, 215M, 245F, 251, 212M, 250, 209F, 222F, 208G, 710LA, 710FS, 730LM, 650AC, and 681 (all manufactured by NEOS Company Limited).

The fluorine-based surfactant has a molecular structure containing a functional group having a fluorine atom, and an acrylic compound in which a portion of the functional group having a fluorine atom is cleaved in a case where heat is applied thereto so that the fluorine atom volatilizes can also be suitably used. Examples of the fluorine-based surfactant include MEGAFACE DS Series (manufactured by DIC Corporation (The Chemical Daily (Feb. 22, 2016) and Nikkei Sangyo Daily (Feb. 23, 2016)) such as MEGAFACE DS-21.

A polymer of a fluorine atom-containing vinyl ether compound containing a fluorinated alkyl group or a fluorinated alkylene ether group and a hydrophilic vinyl ether compound is also preferably used as the fluorine-based surfactant.

A block polymer can also be used as the fluorine-based surfactant.

A fluorine-containing polymer compound having a repeating unit derived from a (meth)acrylate compound having a fluorine atom and a repeating unit derived from a (meth)acrylate compound containing 2 or more (preferably 5 or more) alkyleneoxy groups (preferably ethyleneoxy groups or propyleneoxy groups) can also be used as the fluorine-based surfactant.

A fluorine-containing polymer containing an ethylenically unsaturated bond-containing group in a side chain can also be used as the fluorine-based surfactant. Examples of commercially available products thereof include MEGAFACE RS-101, RS-102, RS-718K, and RS-72-K (all manufactured by DIC Corporation).

Due to concerns of the environmental suitability in a case of a compound containing a linear perfluoroalkyl group having 7 or more carbon atoms, an alternative material for perfluorooctanoic acid (PFOA) or perfluorooctanesulfonic acid (PFOS) is preferably used as the fluorine-based surfactant.

Examples of the silicone-based surfactant include a linear polymer consisting of a siloxane bond and a modified siloxane polymer in which an organic group is introduced into a side chain or a terminal. Examples of commercially available products of the silicone-based surfactant include DOWSIL 8032 ADDITIVE, Toray Silicone DC3PA, Toray Silicone SH7PA, Toray Silicone DC11PA, Toray Silicone SH21PA, Toray Silicone SH28PA, Toray Silicone SH29PA, Toray Silicone SH30PA, and Toray Silicone SH8400 (all manufactured by Dow Toray Co., Ltd.), X-22-4952, X-22-4272, X-22-6266, KF-351A, K354L, KF-355A, KF-945, KF-640, KF-642, KF-643, X-22-6191, X-22-4515, KF-6004, KP-341, KF-6001, and KF-6002 (all manufactured by Shin-Etsu Chemical Co., Ltd.), F-4440, TSF-4300, TSF-4445, TSF-4460, and TSF-4452 (all manufactured by Momentive Performance Materials Inc.), and BYK-307, BYK-323, and BYK-330 (all manufactured by BYK-Chemie GmbH).

Examples of the nonionic surfactant include glycerol, trimethylolpropane, trimethylolethane, ethoxylate and propoxylate thereof (such as glycerol propoxylate or glycerol ethoxylate), polyoxyethylene lauryl ether, polyoxyethylene stearyl ether, polyoxyethylene oleyl ether, polyoxyethylene octyl phenyl ether, polyoxyethylene nonyl phenyl ether, polyethylene glycol dilaurate, polyethylene glycol distearate, and sorbitan fatty acid ester. Examples of commercially available products of the nonionic surfactant include PLURONIC L10, L31, L61, L62, 10R5, 17R2, and 25R2 (all manufactured by BASF SE), TETRONIC 304, 701, 704, 901, 904, and 150R1 (all manufactured by BASF SE), SOLSPERSE 20000 (manufactured by The Lubrizol Corporation), NCW-101, NCW-1001, and NCW-1002 (all manufactured by FUJIFILM Wako Pure Chemical Corporation), PIONIN D-6112, D-6112-W, and D-6315 (all manufactured by Takemoto Oil & Fat Co., Ltd.), and OLFINE E1010, SURFINOL 104, 400, and 440 (all manufactured by Nisshin Chemical Co., Ltd.).

The content of the surfactant in the total solid content of the resin composition is preferably in a range of 0.01% to 3.0% by mass, more preferably in a range of 0.05% to 1.0% by mass, and still more preferably in a range of 0.10% to 0.80% by mass. The surfactant may be used alone or two or more kinds thereof. In a case where two or more kinds of silane coupling agents are used, it is preferable that the total amount is in the above-described range.

<<Solvent>>

It is preferable that the resin composition further contains a solvent. The solvent is not particularly limited, and examples thereof include water and an organic solvent. Examples of the organic solvent include an alcohol-based solvent, an ester-based solvent, a ketone-based solvent, an amide-based solvent, an ether-based solvent, a hydrocarbon-based solvent, and a halogen-based solvent. Specific examples of the organic solvent include methanol, ethanol, 1-propanol, 2-propanol, 1-butanol, 2-butanol, 2-methyl-1-propanol, 1-methoxy-2-propanol, 2-ethoxyethanol, 2-butoxyethanol, polyethylene glycol monoalkyl ether, polypropylene glycol monoalkyl ether, ethylene glycol, propylene glycol, polyethylene glycol, polypropylene glycol, glycerin, ethylene carbonate, N-methylpyrrolidone, dioxane, tetrahydrofuran, ethylene glycol dialkyl ether, propylene glycol dialkyl ether, polyethylene glycol dialkyl ether, polypropylene glycol dialkyl ether, acetonitrile, propionitrile, benzonitrile, carboxylic acid ester, phosphoric acid ester, phosphonic acid ester, dimethyl sulfoxide, sulfolane, dimethylformamide, dimethylacetamide, ethyl acetate, chloroform, methylene chloride, and methyl acetate. The solvent may be used alone or in combination of two or more kinds thereof. The content of the solvent is preferably in a range of 10% to 90% by mass with respect to the total amount of the resin composition.

<<Other Additives>>

The resin composition may appropriately contain optional additives such as an antioxidant, a light stabilizer, a processing stabilizer, an anti-aging agent, and a compatibilizer as necessary. By allowing the resin composition to appropriately contain these components, various characteristics of the cured substance to be obtained can be appropriately adjusted.

<<Applications>>

The resin composition according to the embodiment of the present invention can also be suitably used for applications in a case where the resin composition may be exposed to light including sunlight and ultraviolet rays. Specific examples include coating materials or films for window glass of houses, facilities, and transportation equipment; interior/exterior materials and interior/exterior paints of houses, facilities, and transportation equipment; members for light sources that emit ultraviolet rays, such as a fluorescent lamp and a mercury lamp; solar cells, precision machineries, electronic and electrical equipment, and members for a display device; containers or packaging materials for food, chemicals, and drugs; agricultural and industrial sheets; clothing textile products and fibers such as sportswear, stockings, and hats; lenses such as plastics lenses, contact lenses, glasses, and artificial eyes, or coating materials thereof, optical supplies such as optical filters, prisms, mirrors, and photographic materials; stationery such as tapes and inks; and marking boards, marking devices, and the surface coating materials thereof. For the details thereof, the description in paragraphs 0158 to 0218 of JP2009-263617A can be referred to, and the contents thereof are incorporated in the present specification.

The resin composition according to the embodiment of the present invention can be preferably used for an optical member or the like. The resin composition according to the embodiment of the present invention is preferably used for an ultraviolet cut filter, a lens, a protective material, or the like. The form of the protective material is not particularly limited, and examples thereof include a coating film, a film, and a sheet. Further, the resin composition according to the embodiment of the present invention can also be used as a pressure sensitive adhesive or an adhesive.

Further, the resin composition according to the embodiment of the present invention can also be used for various members of a display device. For example, in a case of a liquid crystal display device, the resin composition can be used for each member constituting the liquid crystal display device such as an antireflection film, a polarizing plate protective film, an optical film, a phase difference film, a pressure sensitive adhesive, and an adhesive. Further, in a case of an organic electroluminescence display device, the polymerizable composition can be used for each member constituting the organic electroluminescence display device such as an optical film, a polarizing plate protective film in a circularly polarizing plate, a phase difference film such as a quarter wave plate, and an adhesive or a pressure sensitive adhesive.

<Cured Substance and Applications>

The cured substance according to the embodiment of the present invention is obtained by using the above-described resin composition according to the embodiment of the present invention. The “cured substance” in the present specification includes a dried product obtained by drying and solidifying the resin composition and a cured substance cured by performing a curing reaction on the resin composition in a case where the resin composition undergoes a curing reaction.

The cured substance according to the embodiment of the present invention may be obtained as a molded product formed by molding the resin composition into a desired shape. The shape of the molded product can be appropriately selected according to the intended use and the purpose. Examples of the shape thereof include a coating film, a film, a sheet, a plate, a lens, a tube, and a fiber.

The cured substance according to the embodiment of the present invention is preferably used as an optical member. Examples of the optical member include an ultraviolet cut filter, a lens, and a protective material. Further, the optical member can also be used as a polarizing plate or the like.

The ultraviolet cut filter can be used for an article such as an optical filter, a display device, a solar cell, or window glass. The kind of display device is not particularly limited, and examples thereof include a liquid crystal display device and an organic electroluminescence display device.

In a case where the cured substance according to the embodiment of the present invention is used for a lens, the cured substance according to the embodiment of the present invention may be formed into a lens shape and used. Further, the cured substance according to the embodiment of the present invention may be used for a coating film on a surface of a lens, an interlayer (adhesive layer) of a cemented lens, or the like. Examples of the cemented lens include those described in paragraphs 0094 to 0102 of WO2019/131572A, and the contents of which are incorporated in the present specification.

The kind of the protective material is not particularly limited, and examples thereof include a protective material for a display device, a protective material for a solar cell, a protective material for window glass, and an organic electroluminescence display device. The shape of the protective material is not particularly limited, and examples thereof include a coating film, a film, and a sheet.

<Optical Member>

An optical member according to the embodiment of the present invention contains the ultraviolet absorbing agent according to the embodiment of the present invention. It is also preferable that the optical member according to the embodiment of the present invention contains a cured substance formed of the resin composition according to the embodiment of the present invention. The cured substance according to the embodiment of the present invention may be obtained as a molded product formed by molding the above-described resin composition according to the embodiment of the present invention into a desired shape. The shape of the molded product can be appropriately selected according to the intended use and the purpose. Examples of the shape thereof include a coating film, a film, a sheet, a plate, a lens, a tube, and a fiber.

In addition, the optical member according to the embodiment of the present invention may be formed of the resin composition according to the embodiment of the present invention. For example, the optical member according to the embodiment of the present invention may be a member obtained by bonding a polarizing plate and a polarizing plate protective film to each other with the resin composition according to the embodiment of the present invention.

Examples of the optical member include an ultraviolet cut filter, a lens, and a protective material.

The ultraviolet cut filter can be used for an article such as an optical filter, a display device, a solar cell, or window glass. The kind of display device is not particularly limited, and examples thereof include a liquid crystal display device and an organic electroluminescence display device.

Examples of the lens include those obtained by forming the cured substance according to the embodiment of the present invention into a lens shape and those obtained by allowing a coating film on a surface of a lens, an interlayer (an adhesive layer or a pressure sensitive adhesive layer) of a cemented lens, or the like to contain the ultraviolet absorbing agent according to the embodiment of the present invention.

The kind of the protective material is not particularly limited, and examples thereof include a protective material for a display device, a protective material for a solar cell, and a protective material for window glass. The shape of the protective material is not particularly limited, and examples thereof include a coating film, a film, and a sheet.

Further, a resin film is exemplified as one form of the optical member. The resin film can be formed of the above-described resin composition according to the embodiment of the present invention. Examples of the resin used in the resin composition for forming a resin film include the above-described resins. Among these, a (meth)acrylic resin, a polyester fiber, a cyclic olefin resin, and a cellulose acylate resin are preferable, and a cellulose acylate resin is more preferable. The resin composition containing the cellulose acylate resin can contain the additives described in paragraphs 0022 to 0067 of JP2012-215689A. Examples of such additives include sugar esters. By adding a sugar ester compound to the resin composition containing a cellulose acylate resin, the total haze and the internal haze can be decreased without impairing the expression of optical properties even in a case where a heat treatment is not performed before a stretching step. Further, the resin film formed of the resin composition containing the cellulose acylate resin (cellulose acylate film) can be produced by the method described in paragraphs 0068 to 0096 of JP2012-215689A. Further, the hard coat layer described in paragraphs 0097 to 0113 of JP2012-215689A may be further laminated on the resin film.

Further, examples of other forms of the optical member include an optical member having a laminate of a support and a resin layer. In this optical member, at least one of the support or the resin layer contains the above-described ultraviolet absorbing agent according to the embodiment of the present invention.

The thickness of the resin layer in the laminate is preferably in a range of 1 m to 2500 m and more preferably in a range of 10 m to 500 m.

A material having transparency within a range where the optical performance is not impaired is preferable as the support in the laminate. The support having transparency denotes that the support is optically transparent and specifically denotes that the total light transmittance of the support is 85% or greater. The total light transmittance of the support is preferably 90% or greater and more preferably 95% or greater.

Suitable examples of the support include a resin film. Examples of the resin constituting a resin film include an ester resin (such as polyethylene terephthalate (PET), polyethylene naphthalate (PEN), polybutylene terephthalate (PBT), or polycyclohexane dimethylene terephthalate (PCT)), an olefin resin (such as polypropylene (PP) or polyethylene (PE)), polyvinyl chloride (PVA), and tricellulose acetate (TAC). Among these, PET is preferable in terms of general purpose properties.

The thickness of the support can be appropriately selected according to the applications, the purpose, and the like. In general, the thickness thereof is preferably in a range of 5 μm to 2,500 μm and more preferably in a range of 20 μm to 500 μm.

In addition, a peelable support can also be used as the support. Such a laminate is preferably used as a polarizing plate or the like. Here, the peelable support denotes a support capable of being peeled off from the ultraviolet shielding material. The stress in a case of peeling the support from the ultraviolet shielding material is preferably in a range of 0.05 N/25 mm or greater and 2.00 N/25 mm or less, more preferably 0.08 N/25 mm or greater and 0.50 N/25 mm or less, and still more preferably 0.11 N/25 mm or greater and 0.20 N/25 mm or less. The stress in a case of peeling the support from the ultraviolet shielding material is evaluated by bonding and fixing the surface of the laminate cut to have a size of a width of 25 mm and a length of 80 mm to a glass base material via an acrylic pressure sensitive adhesive sheet, grasping one end (one side with a width of 25 mm) of a test piece in the length direction using a tension tester (RTF-1210, manufactured by A & D Co., Ltd.), and performing a 90° peeling test (in conformity with Japanese Industrial Standards (JIS) K 6854-1: 1999 “Adhesive-Determination of peel strength of bonded assemblies-Part 1: 90° peel”) in an atmosphere of a temperature of 23° C. and a relative humidity of 60% at a crosshead speed (grasping movement speed) of 200 mm/min.

A support containing polyethylene terephthalate (PET) as a main component (the component having the highest content in terms of mass among the components constituting the support) is preferable as the peelable support. From the viewpoint of mechanical strength, the weight-average molecular weight of PET is preferably 20,000 or greater, more preferably 30,000 or greater, and still more preferably 40,000 or greater. The weight-average molecular weight of PET can be determined by dissolving the support in hexafluoroisopropanol (HFIP) using the above-described GPC method. The thickness of the support is not particularly limited, but is preferably in a range of 0.1 to 100 μm, more preferably in a range of 0.1 to 75 μm, still more preferably in a range of 0.1 to 55 μm, and particularly preferably in a range of 0.1 to 10 μm. Further, the support may be subjected to a corona treatment, a glow discharge treatment, undercoating, or the like as a known surface treatment.

Further, examples of other forms of the optical member include a laminate obtained by laminating a hard coat layer, a transparent support, and a pressure sensitive adhesive layer or an adhesive layer in this order. Such a laminate is preferably used as an ultraviolet cut filter or a protective material (a protective film or a protective sheet). The optical member in this form is not limited as long as any of the support, the hard coat layer, or the pressure sensitive adhesive layer or the adhesive layer contains the above-described ultraviolet shielding material according to the embodiment of the present invention.

As the hard coat layer, any of the hard coat layers described in JP2013-045045A, JP2013-043352A, JP2012-232459A, JP2012-128157A, JP2011-131409A, JP2011-131404A, JP2011-126162A, JP2011-075705A, JP2009-286981A, JP2009-263567A, JP2009-075248A, JP2007-164206A, JP2006-096811A, JP2004-075970A, JP2002-156505A, JP2001-272503A, WO2012/018087A, WO2012/098967A, WO2012/086659A, and WO2011/105594A can be applied. The thickness of the hard coat layer is preferably in a range of 5 μm to 100 μm from the viewpoint of further improving the scratch resistance.

The optical member in this form has a pressure sensitive adhesive layer or an adhesive layer on a side of the support opposite to a side where the hard coat layer is provided. The kind of the pressure sensitive adhesive or the adhesive used for the pressure sensitive adhesive layer or the adhesive layer is not particularly limited, and a known pressure sensitive adhesive or adhesive can be used. As the pressure sensitive adhesive or the adhesive, those containing the acrylic resin described in paragraphs 0056 to 0076 of JP2017-142412A and the crosslinking agent described in paragraphs 0077 to 0082 of JP2017-142412A are also preferably used. Further, the pressure sensitive adhesive or the adhesive may contain the adhesiveness improver (silane compound) described in paragraphs 0088 to 0097 of JP2017-142412A and the additives described in paragraph 0098 of JP2017-142412A. Further, the pressure sensitive adhesive layer or the adhesive layer can be formed by the method described in paragraphs 0099 and 0100 of JP2017-142412A. The thickness of the pressure sensitive adhesive layer or the adhesive layer is preferably in a range of 5 m to 100 m from the viewpoint of achieving both adhesive strength and handleability.

The optical member according to the embodiment of the present invention can be preferably used as a constituent member of a display such as a liquid crystal display device (LCD) or an organic electroluminescence display device (OLED).

Examples of the liquid crystal display device include a liquid crystal display device in which a member such as an antireflection film, a polarizing plate protective film, an optical film, a phase difference film, a pressure sensitive adhesive, or an adhesive contains the ultraviolet shielding material according to the embodiment of the present invention. The optical member containing the ultraviolet shielding material according to the embodiment of the present invention may be disposed on any of a viewer side (front side) or a backlight side with respect to the liquid crystal cell and any of a side far from the liquid crystal cell (outer) or a side close to the liquid crystal cell (inner) with respect to the polarizer.

Examples of the organic electroluminescence display device include an organic electroluminescence display device in which a member such as an optical film, a polarizing plate protective film in a circularly polarizing plate, a phase difference film such as a quarter wave plate, an adhesive, or a pressure sensitive adhesive contains the ultraviolet shielding material according to the embodiment of the present invention. By using the ultraviolet shielding material according to the embodiment of the present invention with the above-described configuration, deterioration of the organic electroluminescence display device due to external light can be suppressed.

<Compound and Method of Synthesizing Compound>

A compound according to the embodiment of the present invention is a compound represented by Formula (1a). The compound represented by Formula (1a) has the same definition as that for the compound described in the section of the ultraviolet absorbing agent above, and the preferable ranges are also the same as described above. The compound represented by Formula (1a) is preferably used as an ultraviolet absorbing agent.

Further, the compound represented by Formula (1a) can be synthesized by reacting the compound represented by Formula (10a) with the compound represented by Formula (20a). Further, it is also preferable that the compound represented by Formula (1a) is synthesized and treated by being brought into contact with an adsorbent. Examples of the adsorbent include the adsorbents described above as being usable in the method of producing the ultraviolet absorbing agent according to the embodiment of the present invention. Among these, activated carbon and activated alumina are preferable, and activated carbon is more preferable.

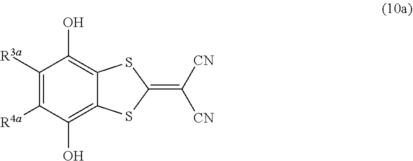

In Formula (10a), R^{3a} represents an alkyl group, and R^{4a} represents a hydrogen atom or an alkyl group.

R^{21a}-E^{21a} (20a)

In Formula (20a), E^{21a} represents a group that reacts with the hydroxy group of Formula (10a), and R^{21a} represents a branched alkyl group having 6 or more carbon atoms. R^{3a} and R^{4a} of Formula (10a) each have the same definition as that for R^{3a} and R^{4a} of Formula (1a). R^{21a} of Formula (20a) has the same definition as that for R^{1a} and R^{2a} of Formula (1a). E^{21a} of Formula (20a) has the same definition as that for E²¹ of Formula (20).

### EXAMPLES

Hereinafter, the present invention will be described in more detail based on the following examples. The materials, the used amounts, the ratios, the treatment contents, the treatment procedures, and the like described in the following examples can be appropriately changed without departing from the gist of the present invention. Therefore, the scope of the present invention is not limited to the following specific examples.

### SYNTHESIS EXAMPLES

### (Synthesis Example 1) Synthesis of Compound A-26

An intermediate 1 was synthesized with reference to the method described in Journal of Chemical Crystallography, (1997), 27 (9), pp. 515 to 526.

3 g of the intermediate 1, 2.78 g of triethylamine, and 30 ml of N,N-dimethylacetamide were added to a flask, mixed, and stirred under ice-cooling for 10 minutes. 2-Ethylhexanoyl chloride was added to the mixed solution in the flask, and the solution was stirred at room temperature for 3 hours. After completion of the reaction, 75 ml of ethyl acetate and 75 ml of hexane were added to the reaction solution, and the organic layer was washed with 60 ml of distilled water three times. Subsequently, the reaction solution was washed with 60 ml of saturated saline and dried over magnesium sulfate. The magnesium sulfate was filtered, and the filtrate was concentrated, thereby obtaining a crude product of a compound A-26. The obtained crude product of the compound A-26 was subjected to a purification treatment using activated carbon (manufactured by FUJIFILM Wako Pure Chemical Corporation) as an adsorbent, thereby obtaining 4.07 g of the compound A-26.

¹H-NMR (CDCl₃): δ 7.17 (s, 1H), 2.62-2.55 (m, 2H), 2.24 (s, 3H), 1.85-1.60 (m, 8H), 1.42-1.37 (m, 8H), 1.09-1.02 (m, 6H), 0.97-0.93 (m, 6H)

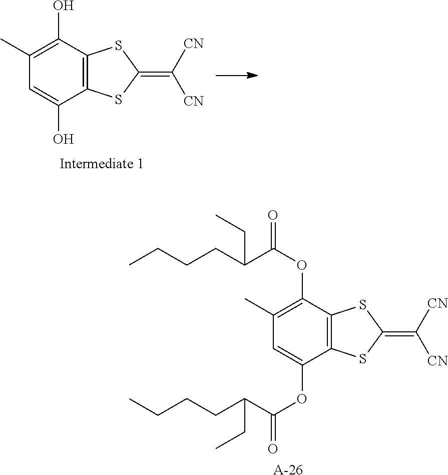

### (Synthesis Example 2) Synthesis of Compound A-28

A crude product of a compound A-28 was obtained by being synthesized by the same method as in Synthesis Example 1 except that diethylcarbamoyl chloride was used in place of 2-ethylhexanoyl chloride in Synthesis Example 1. The obtained crude product of the compound A-28 was subjected to a purification treatment using activated carbon (manufactured by FUJIFILM Wako Pure Chemical Corporation) as an adsorbent, thereby obtaining the compound A-28.

¹H-NMR (CDCl₃): δ 7.22 (d, 1H), 3.48-3.39 (m, 8H), 2.26 (s, 3H), 1.31-1.22 (m, 12H)

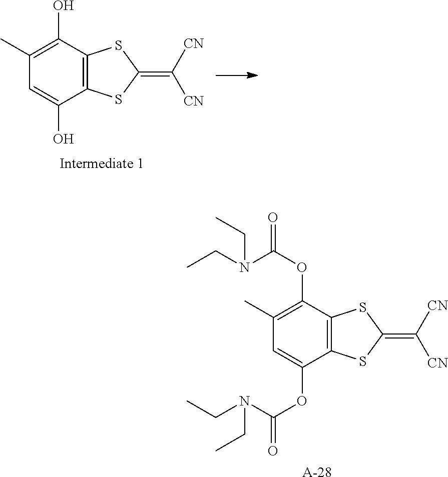

### (Synthesis Example 3) Synthesis of Compound A-35

15 g of the intermediate 1 and 150 ml of N,N-dimethylacetamide were added to a flask, 19 g of potassium carbonate and 24.3 g of 2-ethylhexyl bromide were added thereto, and the mixture was stirred at 80° C. for 4 hours. After completion of the reaction, 150 ml of distilled water was slowly added dropwise thereto, and precipitated crystals were separated by filtration and washed with 150 ml of water. 150 ml of methanol was added to the obtained crystals, and the mixture was stirred for 1 hour while being heated at 80° C. The mixture was cooled at room temperature and stirred for 1 hour, and crystals were filtered and washed with 75 ml of methanol, thereby obtaining 19.85 g of a crude product of a compound A-35.

Subsequently, 200 ml of ethyl acetate and 2 g of activated carbon (manufactured by FUJIFILM Wako Pure Chemical Corporation) as an adsorbent were added to 19.85 g of the crude product of the compound A-35, and the mixture was stirred for 1 hour. The mixture was stirred, and the activated carbon was removed by filtration to obtain a filtrate.

Subsequently, 2 g of activated carbon (manufactured by FUJIFILM Wako Pure Chemical Corporation) was added to the filtrate, the mixture was continuously stirred and filtered three times, and the filtrate was concentrated, thereby obtaining 13.9 g of the compound A-35.

¹H-NMR (CDCl₃): δ 6.69 (s, 1H), 3.97-3.91 (m, 2H), 3.76 (d, 2H), 2.34 (s, 3H) 1.78-1.67 (m, 2H), 1.62-1.29 (m, 16H), 0.99-0.89 (m, 12H)

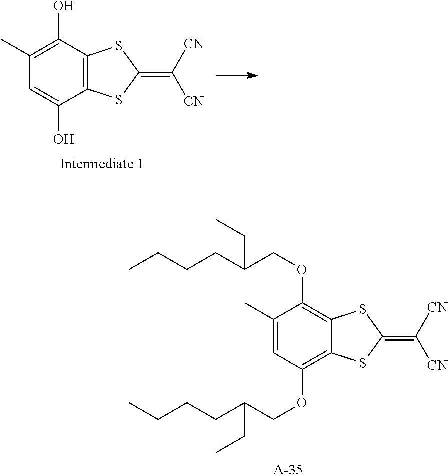 [SMILES: Cc1cc(O)c2c(c1O)SC(=C(C#N)C#N)S2 ; CCCCC(CC)COc1cc(C)c(OCC(CC)CCCC)c2c1SC(=C(C#N)C#N)S2 ; "A-35"]

### (Synthesis Example 4) Synthesis of Compound A-30

A crude product of a compound A-30 was obtained by the same method as in Synthesis Example 3 except that 2-ethylhexyl bromide was changed to methyl bromoacetate in Synthesis Example 3.

Subsequently, 100 ml of ethyl acetate and 1 g of activated carbon (manufactured by FUJIFILM Wako Pure Chemical Corporation) as an adsorbent were added to 10 g of the crude product of the compound A-30, and the mixture was stirred at room temperature for 1 hour. The mixture was stirred, and the activated carbon was removed by filtration to obtain a filtrate. 1 g of activated carbon was added to the filtrate, and the mixture was stirred at room temperature for 1 hour. The mixture was stirred, and the activated carbon was removed by filtration to obtain a filtrate. Subsequently, 1 g of activated alumina was added to the filtrate as an adsorbent, and the mixture was stirred at room temperature for 1 hour. After the stirring, the activated alumina was removed by filtration, and the filtrate was concentrated, thereby obtaining a compound A-30.

¹H-NMR (CDCl₃): δ 6.60 (s, 1H), 4.74 (s, 2H), 4.54 (s, 2H), 3.85 (s, 3H), 3.83 (s, 3H), 2.36 (s, 3H)

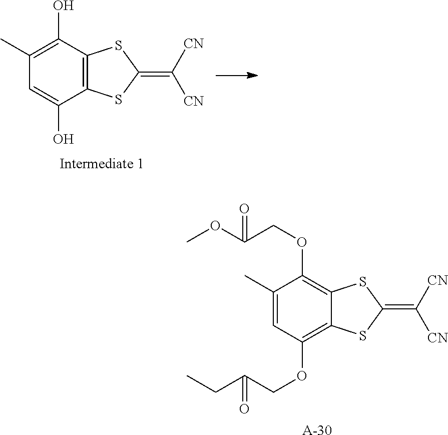 [SMILES: Cc1cc(O)c2c(c1O)SC(=C(C#N)C#N)S2 ; CCC(=O)COc1cc(C)c(OCC(=O)OC)c2c1SC(=C(C#N)C#N)S2 ; "A-30"]

### (Synthesis Example 5) Synthesis of Compound A-31

A crude product of a compound A-31 was obtained by the same method as in Synthesis Example 3 except that 2-ethylhexyl bromide was changed to methyl 2-bromopropionate in Synthesis Example 3.

Subsequently, 100 ml of ethyl acetate and 1 g of activated carbon (manufactured by FUJIFILM Wako Pure Chemical Corporation) as an adsorbent were added to 1 g of the crude product of the compound A-31, and the mixture was stirred at room temperature for 1 hour. The mixture was stirred, and the activated carbon was removed by filtration to obtain a filtrate. 1 g of activated carbon was added to the filtrate, and the mixture was stirred at room temperature for 1 hour. The mixture was stirred, the activated carbon was removed by filtration, and the filtrate was concentrated, thereby obtaining the compound A-31.

¹H-NMR (CDCl₃): δ 6.56 (s, 1H), 4.85-4.80 (m, 1H), 4.65-4.60 (m, 1H), 3.78 (s, 6H), 2.32 (s, 3H), 1.66 (d, 3H), 1.61 (d, 3H)

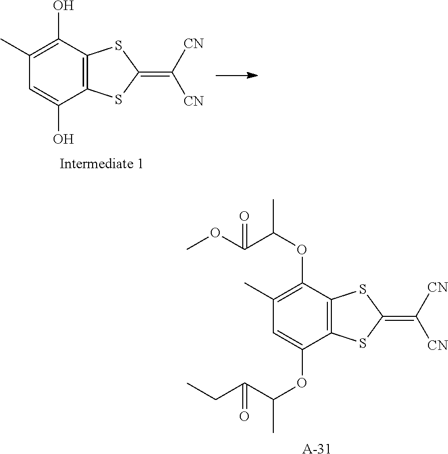

### (Synthesis Example 6) Synthesis of Compound A-48

A crude product of a compound A-48 was synthesized by the same method as in Synthesis Example 4 except that the intermediate 2 was used in place of the intermediate 1 in Synthesis Example 4.

Subsequently, 100 ml of ethyl acetate and 1 g of activated carbon (manufactured by FUJIFILM Wako Pure Chemical Corporation) as an adsorbent were added to 10 g of the crude product of the compound A-48, and the mixture was stirred at room temperature for 1 hour. The mixture was stirred, and the activated carbon was removed by filtration to obtain a filtrate. 1 g of activated carbon was added to the filtrate, and the mixture was stirred at room temperature for 1 hour. The mixture was stirred, and the activated carbon was removed by filtration to obtain a filtrate. Subsequently, 1 g of activated carbon was added to the filtrate as an adsorbent, and the mixture was stirred at room temperature for 1 hour. After the stirring, the activated alumina was removed by filtration, and the filtrate was concentrated, thereby obtaining a compound A-48.

¹H-NMR (CDCl₃): δ 6.80 (s, 1H), 4.76 (s, 2H), 4.57 (s, 2H), 3.89 (s, 3H), 3.83 (s, 3H), 1.40 (s, 9H)

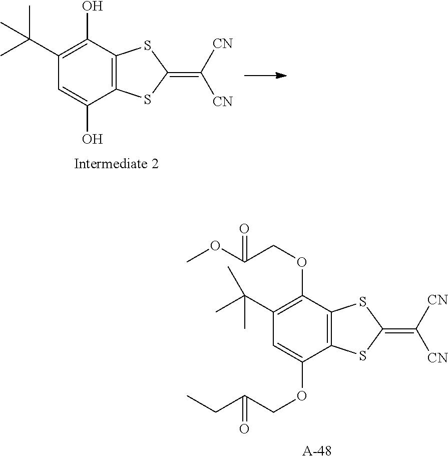 [SMILES: CC(C)(C)c1cc(O)c2c(c1O)SC(=C(C#N)C#N)S2 ; CCC(=O)COc1cc(C(C)(C)C)c(OCC(=O)OC)c2c1SC(=C(C#N)C#N)S2 ; "A-48"]

### (Synthesis Example 7) Synthesis of Compound A-37

A crude product of a compound A-37 was synthesized by the same method as in Synthesis Example 3 except that 2-ethylhexyl bromide was changed to 1-bromopentane in Synthesis Example 3.

Subsequently, 100 ml of ethyl acetate and 1 g of activated carbon (manufactured by FUJIFILM Wako Pure Chemical Corporation) as an adsorbent were added to 10 g of the crude product of the compound A-37, and the mixture was stirred at room temperature for 1 hour. The mixture was stirred, and the activated carbon was removed by filtration to obtain a filtrate.

Subsequently, 2 g of activated carbon (manufactured by FUJIFILM Wako Pure Chemical Corporation) was added to the filtrate, the mixture was continuously stirred and filtered two times, and the filtrate was concentrated, thereby obtaining a compound A-37.

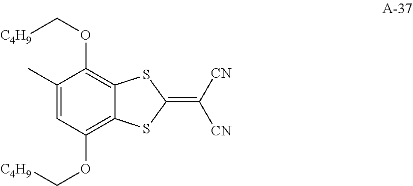

### (Synthesis Examples 8 to 22) Synthesis of Compounds A-1, A-3, A-12, A-13, A-14, A-17, A-19, A-36, A-40, A-42, A-45, A-47, A-59, A-65, and A-67

Crude products of compounds A-1, A-3, A-12, A-13, A-14, A-17, A-19, A-36, A-40, A-42, A-45, A-47, A-59, A-65, and A-67 were synthesized by the same methods as in Synthesis Examples 1 to 7.

Subsequently, 100 ml of ethyl acetate and 1 g of activated carbon (manufactured by FUJIFILM Wako Pure Chemical Corporation) as an adsorbent were added to 10 g of the crude product of each compound, and the mixture was stirred at room temperature for 1 hour. The mixture was stirred, and the activated carbon was removed by filtration to obtain a filtrate.

Subsequently, 2 g of activated carbon (manufactured by FUJIFILM Wako Pure Chemical Corporation) was added to the filtrate, the mixture was continuously stirred and filtered two times, and the filtrate was concentrated, thereby obtaining each of compounds A-1, A-3, A-12, A-13, A-14, A-17, A-19, A-36, A-40, A-42, A-45, A-47, A-59, A-65, and A-67.

### (Synthesis Example 23) Synthesis of Compound A-71

A crude product of a compound A-71 was synthesized by the same method as in Synthesis Example 3 except that 2-ethylhexyl bromide was changed to ethyl 4-bromobutyrate in Synthesis Example 3.

Subsequently, 50 ml of ethyl acetate and 0.5 g of activated carbon (manufactured by FUJIFILM Wako Pure Chemical Corporation) as an adsorbent were added to 5 g of the crude product of the compound A-71, and the mixture was stirred at room temperature for 1 hour. The mixture was stirred, the activated carbon was removed by filtration, and the filtrate was concentrated, thereby obtaining the compound A-71.

¹H-NMR (CDCl₃): δ 6.70 (s, 1H), 4.18 (q, 4H), 4.16 (t, 2H), 3.93 (t, 2H), 2.56 (t, 2H), 2.51 (t, 2H), 2.32 (s, 3H), 2.14 (m, 4H), 2.51 (t, 2H), 1.28 (t, 6H)

### (Synthesis Example 24) Synthesis of Compound A-73

A crude product of a compound A-73 was synthesized by the same method as in Synthesis Example 3 except that 2-ethylhexyl bromide was changed to ethyl 5-bromovalerate in Synthesis Example 3.

Subsequently, 50 ml of ethyl acetate and 0.5 g of activated carbon (manufactured by FUJIFILM Wako Pure Chemical Corporation) as an adsorbent were added to 5 g of the crude product of the compound A-73, and the mixture was stirred at room temperature for 1 hour. The mixture was stirred, the activated carbon was removed by filtration, and the filtrate was concentrated, thereby obtaining the compound A-73.

MS: m/z=519 (M⁺, 100%)

### (Synthesis Example 25) Synthesis of Compound A-78

A crude product of a compound A-78 was synthesized by the same method as in Synthesis Example 3 except that 2-ethylhexyl bromide was changed to ethyl 6-bromohexanoate in Synthesis Example 3.

Subsequently, 50 ml of ethyl acetate and 0.5 g of activated carbon (manufactured by FUJIFILM Wako Pure Chemical Corporation) as an adsorbent were added to 5 g of the crude product of the compound A-78, and the mixture was stirred at room temperature for 1 hour. The mixture was stirred, the activated carbon was removed by filtration, and the filtrate was concentrated, thereby obtaining the compound A-78.

MS: m/z=547 (M⁺, 100%)

### (Synthesis Example 26) Synthesis of Compound A-81

A crude product of a compound A-81 was synthesized by the same method as in Synthesis Example 3 except that 2-ethylhexyl bromide was changed to ethyl 11-bromoundecanoate in Synthesis Example 3.

Subsequently, 50 ml of ethyl acetate and 0.5 g of activated carbon (manufactured by FUJIFILM Wako Pure Chemical Corporation) as an adsorbent were added to 5 g of the crude product of the compound A-81, and the mixture was stirred at room temperature for 1 hour. The mixture was stirred, the activated carbon was removed by filtration, and the filtrate was concentrated, thereby obtaining the compound A-81.

MS: m/z=687 (M⁺, 100%)

### (Synthesis Example 27) Synthesis of Compound A-82

A crude product of a compound A-82 was synthesized by the same method as in Synthesis Example 3 except that 2-ethylhexyl bromide was changed to tert-butyl 4-bromobutanoate in Synthesis Example 3.

Subsequently, 50 ml of ethyl acetate and 0.5 g of activated carbon (manufactured by FUJIFILM Wako Pure Chemical Corporation) as an adsorbent were added to 5 g of the crude product of the compound A-82, and the mixture was stirred at room temperature for 1 hour. The mixture was stirred, the activated carbon was removed by filtration, and the filtrate was concentrated, thereby obtaining the compound A-82.

MS: m/z=547 (M⁺, 100%)

### (Synthesis Example 28) Synthesis of Compound A-83

A crude product of a compound A-83 was synthesized by the same method as in Synthesis Example 3 except that 2-ethylhexyl bromide was changed to 3-phenoxypropyl bromide in Synthesis Example 3.

Subsequently, 50 ml of ethyl acetate and 0.5 g of activated carbon (manufactured by FUJIFILM Wako Pure Chemical Corporation) as an adsorbent were added to 5 g of the crude product of the compound A-83, and the mixture was stirred at room temperature for 1 hour. The mixture was stirred, the activated carbon was removed by filtration, and the filtrate was concentrated, thereby obtaining the compound A-83.

MS: m/z=531 (M⁺, 100%)

### (Synthesis Example 29) Synthesis of Compound A-84

A crude product of a compound A-84 was synthesized by the same method as in Synthesis Example 3 except that 2-ethylhexyl bromide was changed to diethyl (5-bromopentyl) malonate in Synthesis Example 3.

Subsequently, 50 ml of ethyl acetate and 0.5 g of activated carbon (manufactured by FUJIFILM Wako Pure Chemical Corporation) as an adsorbent were added to 5 g of the crude product of the compound A-84, and the mixture was stirred at room temperature for 1 hour. The mixture was stirred, the activated carbon was removed by filtration, and the filtrate was concentrated, thereby obtaining the compound A-84.

MS: m/z=733 (M⁺, 100%)

### (Synthesis Example 30) Synthesis of Compound A-88

A crude product of a compound A-88 was synthesized by the same method as in Synthesis Example 3 except that 2-ethylhexyl bromide was changed to butyl 4-bromobutyrate in Synthesis Example 3.

Subsequently, 50 ml of ethyl acetate and 0.5 g of activated carbon (manufactured by FUJIFILM Wako Pure Chemical Corporation) as an adsorbent were added to 5 g of the crude product of the compound A-88, and the mixture was stirred at room temperature for 1 hour. The mixture was stirred, the activated carbon was removed by filtration, and the filtrate was concentrated, thereby obtaining the compound A-88.

MS: m/z=547 (M⁺, 100%)

### (Synthesis Example 31) Synthesis of Compound A-92

A crude product of a compound A-92 was synthesized by the same method as in Synthesis Example 3 except that 2-ethylhexyl bromide was changed to 2-ethylhexyl 4-bromobutyrate in Synthesis Example 3.

Subsequently, 50 ml of ethyl acetate and 0.5 g of activated carbon (manufactured by FUJIFILM Wako Pure Chemical Corporation) as an adsorbent were added to 5 g of the crude product of the compound A-92, and the mixture was stirred at room temperature for 1 hour. The mixture was stirred, the activated carbon was removed by filtration, and the filtrate was concentrated, thereby obtaining the compound A-92.

MS: m/z=659 (M⁺, 100%)

### (Synthesis Example 32) Synthesis of Compound A-93

A crude product of a compound A-93 was synthesized by the same method as in Synthesis Example 6 except that methyl bromoacetate was changed to ethyl 4-bromobutyrate in Synthesis Example 6.

Subsequently, 50 ml of ethyl acetate and 0.5 g of activated carbon (manufactured by FUJIFILM Wako Pure Chemical Corporation) as an adsorbent were added to 5 g of the crude product of the compound A-93, and the mixture was stirred at room temperature for 1 hour. The mixture was stirred, the activated carbon was removed by filtration, and the filtrate was concentrated, thereby obtaining the compound A-93.

MS: m/z=533 (M⁺, 100%)

### (Synthesis Example 33) Synthesis of Compound A-94

A crude product of a compound A-94 was synthesized by the same method as in Synthesis Example 6 except that methyl bromoacetate was changed to butyl 4-bromobutyrate in Synthesis Example 6.

Subsequently, 50 ml of ethyl acetate and 0.5 g of activated carbon (manufactured by FUJIFILM Wako Pure Chemical Corporation) as an adsorbent were added to 5 g of the crude product of the compound A-94, and the mixture was stirred at room temperature for 1 hour. The mixture was stirred, the activated carbon was removed by filtration, and the filtrate was concentrated, thereby obtaining the compound A-94.

MS: m/z=589 (M⁺, 100%)

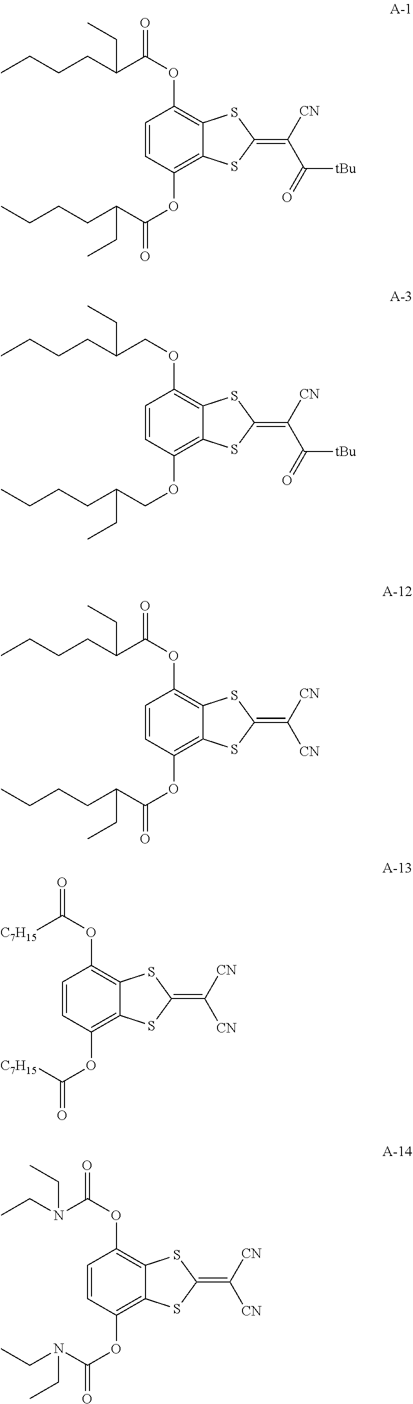

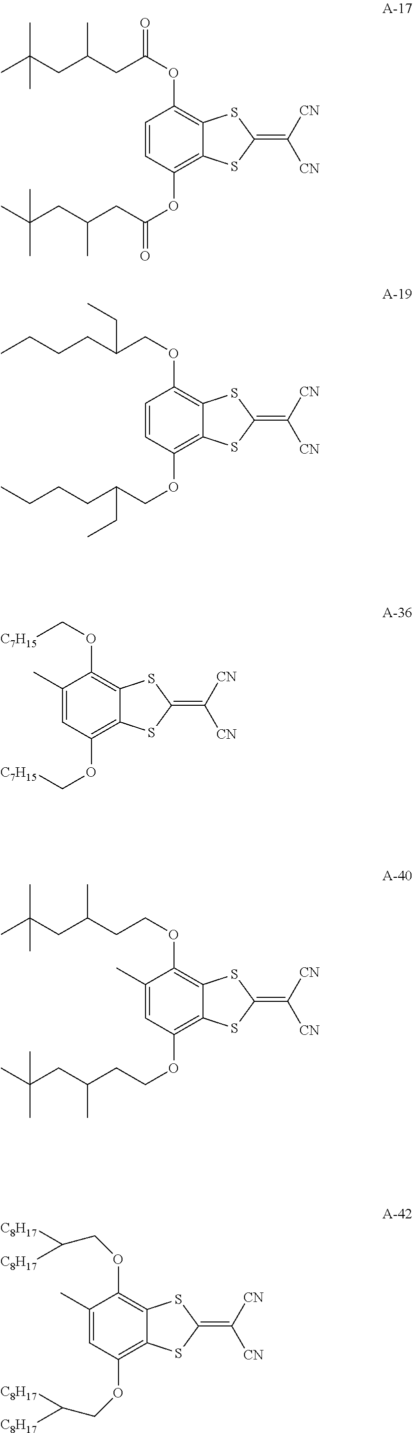

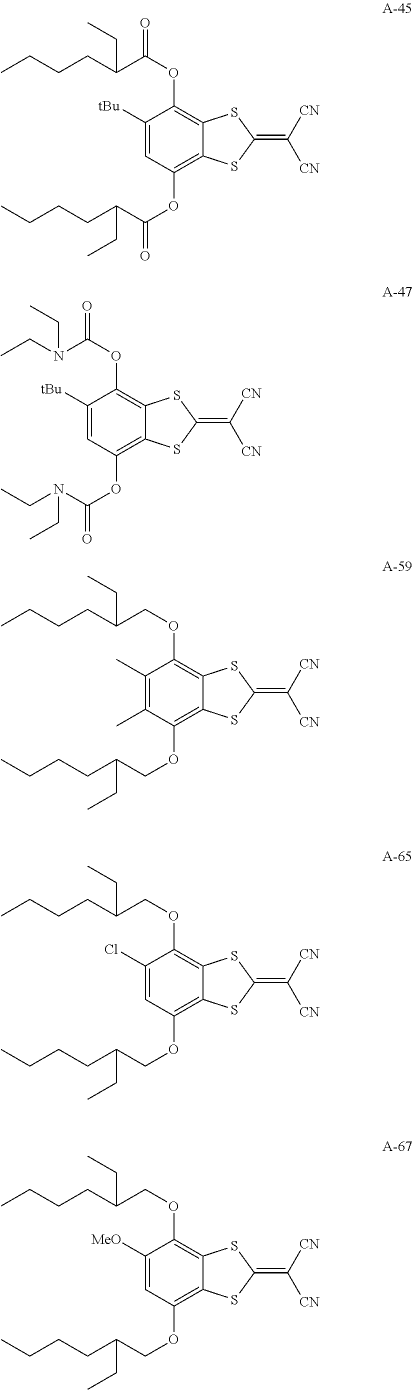

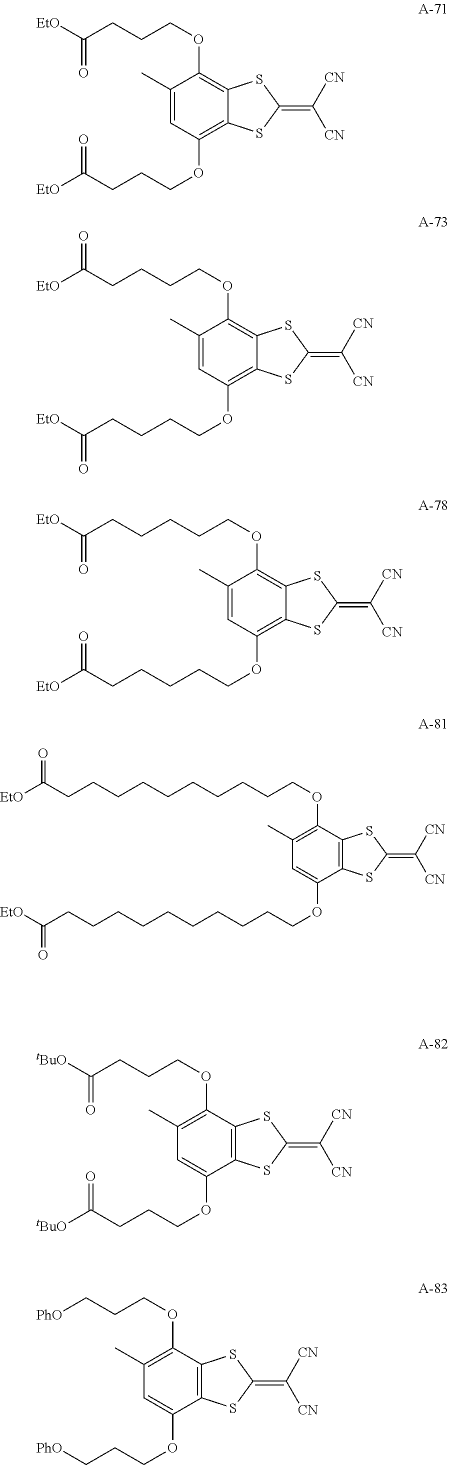 [SMILES: CCOC(=O)CCCOc1cc(C)c(OCCCC(=O)OCC)c2c1SC(=C(C#N)C#N)S2 ; CCOC(=O)CCCCOc1cc(C)c(OCCCCC(=O)OCC)c2c1SC(=C(C#N)C#N)S2 ; CCOC(=O)CCCCCOc1cc(C)c(OCCCCCC(=O)OCC)c2c1SC(=C(C#N)C#N)S2 ; CCOC(=O)CCCCCCCCCCOc1cc(C)c(OCCCCCCCCCCC(=O)OCC)c2c1SC(=C(C#N)C#N)S2 ; Cc1cc(OCCCC(=O)OC(C)(C)C)c2c(c1OCCCC(=O)OC(C)(C)C)SC(=C(C#N)C#N)S2 ; Cc1cc(OCCCOc2ccccc2)c2c(c1OCCCOc1ccccc1)SC(=C(C#N)C#N)S2]

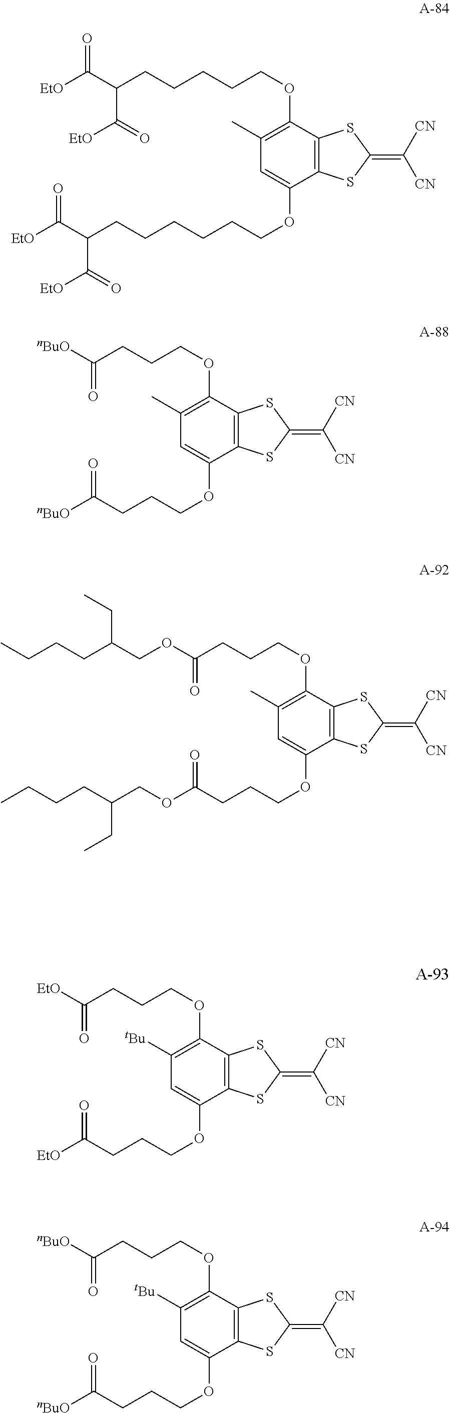

<Evaluation of Solution Spectrum>

A sample solution was prepared by dissolving 2 mg of the compounds listed in the following table in 100 mL of ethyl acetate and diluting the solution with ethyl acetate so that the absorbance of the solution was in a range of 0.6 to 1.2.

The absorbance of each sample solution was measured in a 1 cm quartz cell using a spectrophotometer UV-1800PC (manufactured by Shimadzu Corporation). The maximum absorption wavelength (λ max) was measured from the absorption spectrum of each sample solution. The value of λ max of each compound and the value obtained by dividing the absorbance at a wavelength of 430 nm by the absorbance at the maximum absorption wavelength (absorbance ratio 1) are listed in the following table.

In addition, as the compounds used in Examples 1 to 31, the compounds obtained in the synthesis examples described above, that is, the compounds obtained by treating the crude products of the compounds with an adsorbent and purifying the crude products were used. Further, as the crude product of the compound A-37 used in Comparative Example 1, the crude material in a state before being treated with an adsorbent in Synthesis Example 7 was used.

TABLE 1

| | | Maximum absorption | Absorbance |
|---|---|---|---|
| | Compound | wavelength (nm) | ratio 1 |
| Example 1 | A-1 | 375 | 0.002 |
| Example 2 | A-3 | 381 | 0.001 |
| Example 3 | A-12 | 362 | 0.001 |
| Example 4 | A-13 | 362 | 0.001 |
| Example 5 | A-14 | 364 | 0.001 |
| Example 6 | A-17 | 362 | 0.001 |
| Example 7 | A-19 | 368 | <0.001 |
| Example 8 | A-26 | 366 | <0.001 |
| Example 9 | A-28 | 368 | <0.001 |
| Example 10 | A-35 | 372 | <0.001 |
| Example 11 | A-36 | 372 | <0.001 |
| Example 12 | A-37 | 372 | <0.001 |
| Example 13 | A-40 | 372 | <0.001 |
| Example 14 | A-42 | 372 | <0.001 |
| Example 15 | A-45 | 366 | <0.001 |
| Example 16 | A-47 | 368 | <0.001 |
| Example 17 | A-48 | 370 | 0.001 |
| Example 18 | A-59 | 372 | <0.001 |
| Example 19 | A-65 | 368 | <0.001 |
| Example 20 | A-67 | 377 | <0.001 |
| Example 21 | A-71 | 372 | <0.001 |
| Example 22 | A-73 | 372 | <0.001 |
| Example 23 | A-78 | 372 | <0.001 |
| Example 24 | A-81 | 372 | <0.001 |
| Example 25 | A-82 | 372 | <0.001 |
| Example 26 | A-83 | 372 | <0.001 |
| Example 27 | A-84 | 372 | <0.001 |
| Example 28 | A-88 | 372 | <0.001 |
| Example 29 | A-92 | 372 | <0.001 |
| Example 30 | A-93 | 372 | <0.001 |
| Example 31 | A-94 | 372 | <0.001 |
| Comparative | Crude | 372 | 0.035 |
| Example 1 | product | | |
| | of A-37 | | |

### Examples 101 to 131 and Comparative Example 101

A resin composition was prepared by mixing the compound listed in the following table as an ultraviolet absorbing agent, 7.6 g of chloroform as a solvent, and 1.1 g of a (meth)acrylic resin (DIANAL BR-80, manufactured by Mitsubishi Chemical Corporation). A glass substrate was spin-coated with the obtained resin composition and dried at 40° C. for 2 minutes, thereby producing a resin film. As the compounds used in Examples 101 to 131, the compounds obtained in the synthesis examples described above, that is, the compounds obtained by treating the crude products of the compounds with an adsorbent and purifying the crude products were used. Further, as the crude product of the compound A-37 used in Comparative Example 101, the crude material in a state before being treated with an adsorbent in Synthesis Example 7 was used.

(Measurement of Fluorescence Intensity)

With respect to the obtained resin film, the emission spectrum obtained from the absorption maximum wavelength on a long wavelength side was measured using the absorption maximum wavelength as the excitation wavelength with a fluorescence spectrometer “Spectrophotometer F-7100, manufactured by Hitachi, Ltd.”, and the maximum fluorescence wavelength and the fluorescence intensity at the maximum fluorescence wavelength were measured.

(Evaluation of Light Resistance)

A light resistance test was performed on the obtained resin film using a low-temperature cycle xenon weather meter (Suga tester: XL75) under the conditions of an irradiation amount of 10 klx (40 w/m²), a temperature of 23° C., and a relative humidity of 50% for 24 hours.

The absorbance of the resin film before the light resistance test at the absorption maximum wavelength and the absorbance of the resin film after the light resistance test at the maximum absorption wavelength were measured, the residual rate was calculated by the following equation, and the light resistance was evaluated. The light resistance is more excellent as the residual rate increases.

Residual rate %=((absorbance of λ max after light resistance test)/(absorbance of resin film before light resistance test at absorption maximum wavelength))×100

TABLE 2

| | | | Evaluation of | | |
|---|---|---|---|---|---|
| | Ultraviolet | | fluorescence intensity | | |
| | absorbing agent | | | Fluorescence | Evaluation |
| | | Addi- | Maximum | intensity at | of light |
| | | tion | fluorescence | maximum | resistance |
| | | amount | wavelength | absorption | Residual |
| | Type | (mg) | (nm) | wavelength | rate (%) |
| Example 101 | A-1 | 15.6 | No peaks | — | 91 |
| Example 102 | A-3 | 14.9 | No peaks | — | 92 |
| Example 103 | A-12 | 14.0 | No peaks | — | 92 |
| Example 104 | A-13 | 14.0 | No peaks | — | 89 |
| Example 105 | A-14 | 12.5 | No peaks | — | 90 |
| Example 106 | A-17 | 14.8 | No peaks | — | 91 |
| Example 107 | A-19 | 13.2 | No peaks | — | 92 |
| Example 108 | A-26 | 14.4 | No peaks | — | 93 |
| Example 109 | A-28 | 12.9 | No peaks | — | 90 |
| Example 110 | A-35 | 13.6 | No peaks | — | 90 |
| Example 111 | A-36 | 13.6 | No peaks | — | 91 |
| Example 112 | A-37 | 11.2 | No peaks | — | 91 |
| Example 113 | A-40 | 14.4 | No peaks | — | 89 |
| Example 114 | A-42 | 21.4 | No peaks | — | 92 |
| Example 115 | A-45 | 15.6 | No peaks | — | 89 |
| Example 116 | A-47 | 14.0 | No peaks | — | 91 |
| Example 117 | A-48 | 12.5 | No peaks | — | 89 |
| Example 118 | A-59 | 14.0 | No peaks | — | 90 |
| Example 119 | A-65 | 14.2 | No peaks | — | 91 |
| Example 120 | A-67 | 14.0 | No peaks | — | 90 |
| Example 121 | A-71 | 11.2 | No peaks | — | 90 |
| Example 122 | A-73 | 12.7 | No peaks | — | 91 |
| Example 123 | A-78 | 14.5 | No peaks | — | 90 |
| Example 124 | A-81 | 19.2 | No peaks | — | 90 |
| Example 125 | A-82 | 15.3 | No peaks | — | 91 |
| Example 126 | A-83 | 14.8 | No peaks | — | 88 |
| Example 127 | A-84 | 20.5 | No peaks | — | 90 |
| Example 128 | A-88 | 15.3 | No peaks | — | 91 |
| Example 129 | A-92 | 16.8 | No peaks | — | 90 |
| Example 130 | A-93 | 13.7 | No peaks | — | 90 |
| Example 131 | A-94 | 16.5 | No peaks | — | 89 |
| Comparative | Crude | 11.2 | 451 | 0.16 | 82 |
| Example 101 | product | | | | |
| | of A-37 | | | | |

As listed in the table above, the resin films of Examples 101 to 131 had excellent light resistance. Further, the fluorescence intensity was extremely low, which was below the detection limit value.

### Examples 201 to 231 and Comparative Example 201

A resin composition was prepared by mixing the compound listed in the following table as an ultraviolet absorbing agent, 7.6 g of a mixed solution of ethyl acetate and hexane at a volume ratio of 4/1 as a solvent, and 1.1 g of a (meth)acrylic resin (DIANAL BR-80, manufactured by Mitsubishi Chemical Corporation). A glass substrate was spin-coated with the obtained resin composition and dried at 40° C. for 2 minutes, thereby producing a resin film. As the compounds used in Examples 201 to 231, the compounds obtained in the synthesis examples described above, that is, the compounds obtained by treating the crude products of the compounds with an adsorbent and purifying the crude products were used. Further, as the crude product of the compound A-37 used in Comparative Example 201, the crude material in a state before being treated with an adsorbent in Synthesis Example 7 was used.

The obtained resin film was observed with an optical microscope (MX-61L, manufactured by Olympus Corporation) at a bright field of view of 200 magnifications, and the resin film was observed to confirm the presence of unevenness. In a case where the film was uniform with no unevenness confirmed by an optical microscope, it is determined that the film has excellent resistance to the thermal stress during film formation.

A: Unevenness was not found

B: Unevenness was slightly found

C: Unevenness was significant

TABLE 3

| | Ultraviolet absorbing agent | | Evaluation |
|---|---|---|---|
| | | Addition | of surface |
| | Type | amount (mg) | unevenness |
| Example 201 | A-1 | 15.6 | B |
| Example 202 | A-3 | 14.9 | B |
| Example 203 | A-12 | 14.0 | B |
| Example 204 | A-13 | 14.0 | B |
| Example 205 | A-14 | 12.5 | B |
| Example 206 | A-17 | 14.8 | B |
| Example 207 | A-19 | 13.2 | B |
| Example 208 | A-26 | 14.4 | A |
| Example 209 | A-28 | 12.9 | A |
| Example 210 | A-35 | 13.6 | A |
| Example 211 | A-36 | 13.6 | B |
| Example 212 | A-37 | 11.2 | B |
| Example 213 | A-40 | 14.4 | A |
| Example 214 | A-42 | 21.4 | A |
| Example 215 | A-45 | 15.6 | A |
| Example 216 | A-47 | 14.0 | A |
| Example 217 | A-48 | 12.5 | A |
| Example 218 | A-59 | 14.0 | A |
| Example 219 | A-65 | 14.2 | A |
| Example 220 | A-67 | 14.0 | A |
| Example 221 | A-71 | 11.2 | A |
| Example 222 | A-73 | 12.7 | A |
| Example 223 | A-78 | 14.5 | A |
| Example 234 | A-81 | 19.2 | A |
| Example 225 | A-82 | 15.3 | A |
| Example 226 | A-83 | 14.8 | A |
| Example 227 | A-84 | 20.5 | A |
| Example 228 | A-88 | 15.3 | A |
| Example 229 | A-92 | 16.8 | A |
| Example 230 | A-93 | 13.7 | A |
| Example 231 | A-94 | 16.5 | A |
| Comparative | Crude | 11.2 | B |
| Example 201 | product | | |
| | of A-37 | | |

In all the examples, the surface unevenness was not found or was slightly found.

What is claimed is:

1. An ultraviolet absorbing agent comprising:
a compound represented by Formula (1),
wherein the ultraviolet absorbing agent has a maximum absorption wavelength in a wavelength range of 350 to 390 nm in an ethyl acetate solution, and a value obtained by dividing an absorbance at a wavelength of 430 nm by an absorbance at the maximum absorption wavelength is 0.01 or less, 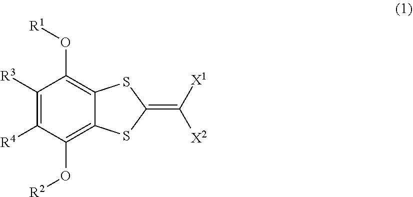
in Formula (1), X¹ and X² each independently represent a hydrogen atom or a substituent,
R¹ and R² each independently represent an alkyl group, an acyl group, a carbamoyl group, an aryl group, an alkoxycarbonyl group, or an aryloxycarbonyl group, and
R³ and R⁴ each independently represent a hydrogen atom, a halogen atom, an alkyl group, an aryl group, an alkoxy group, or an aryloxy group, where at least one of X¹ or X² represents a substituent having a Hammett's substituent constant σp value of 0.2 or greater.

2. The ultraviolet absorbing agent according to claim 1,
wherein X¹ and X² in Formula (1) represent a cyano group.

3. The ultraviolet absorbing agent according to claim 1,
wherein R¹ and R² in Formula (1) each independently represent a branched alkyl group having 6 or more carbon atoms, and at least one of R³ or R⁴ represents an alkyl group, an alkoxy group, or an aryloxy group.

4. The ultraviolet absorbing agent according to claim 1,
wherein R¹ and R² in Formula (1) each independently represent a branched alkyl group having 6 or more carbon atoms, R³ represents an alkyl group, and R⁴ represents a hydrogen atom or an alkyl group.

5. A resin composition comprising:
the ultraviolet absorbing agent according to claim 1; and
a resin.

6. The resin composition according to claim 5,
wherein the resin is at least one selected from a (meth)acrylic resin, a polystyrene resin, a polyester resin, a polyurethane resin, a thiourethane resin, a polyimide resin, an epoxy resin, a polycarbonate resin, or a cellulose acylate resin.

7. A cured substance which is formed of the resin composition according to claim 5.

8. An optical member comprising:
the ultraviolet absorbing agent according to claim 1.

9. A method of producing the ultraviolet absorbing agent according to claim 1, the method comprising:
reacting a compound represented by Formula (10) with a compound represented by Formula (20) to synthesize a compound represented by Formula (1) and treating the synthesized compound by bringing the compound into contact with an adsorbent, 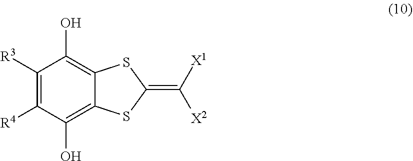
in Formula (10), X¹ and X² each independently represent a hydrogen atom or a substituent, and
R³ and R⁴ each independently represent a hydrogen atom, a halogen atom, an alkyl group, an aryl group, an alkoxy group, or an aryloxy group, where at least one of X¹ or X² represents a substituent having a Hammett's substituent constant σp value of 0.2 or greater,
R²¹-E²¹ (20)
in Formula (20), E²¹ represents a group that reacts with a hydroxy group in Formula (10), and R²¹ represents an alkyl group, an acyl group, a carbamoyl group, an aryl group, an alkoxycarbonyl group, or an aryloxycarbonyl group, 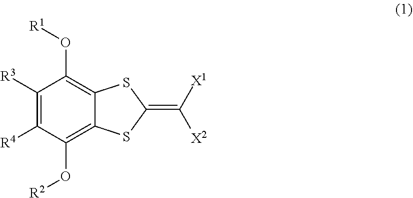
in Formula (1), X¹ and X² each independently represent a hydrogen atom or a substituent,
R¹ and R² each independently represent an alkyl group, an acyl group, a carbamoyl group, an aryl group, an alkoxycarbonyl group, or an aryloxycarbonyl group, and
R³ and R⁴ each independently represent a hydrogen atom, a halogen atom, an alkyl group, an aryl group, an alkoxy group, or an aryloxy group, where at least one of X¹ or X² represents a substituent having a Hammett's substituent constant σp value of 0.2 or greater.

10. The method of producing an ultraviolet absorbing agent according to claim 9,
wherein the adsorbent is at least one selected from activated carbon or activated alumina.

11. A compound which is represented by Formula (1a), 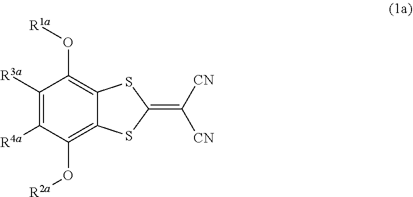
in Formula (1a), R^{1a} and R^{2a} each independently represent a branched alkyl group having 6 or more carbon atoms,
R^{3a} represents an alkyl group, and
R^{4a} represents a hydrogen atom or an alkyl group.
